(19) United States
(12) Patent Application Publication
(10) Pub. No.: US 2026/0283922 A1
(43) Pub. Date: Sep. 24, 2026

(54) Particles of Hydrophobic-Chain-Modified Monosaccharides and Cyclodextrin

(71) Applicants: PARIS SCIENCES ET LETTRES, PARIS (FR); ECOLE NATIONALE SUPERIEURE DE CHIMIE DE PARIS, PARIS CEDEX 05 (FR); ECOLE NORMALE SUPERIEURE, PARIS (FR); CENTRE NATIONAL DE LA RECHERCHE SCIENTIFIQUE (CNRS), PARIS (FR); SORBONNE UNIVERSITE, PARIS (FR); UNIVERSITE PARIS-SACLAY, PARIS (FR)

(72) Inventors: Kawthar BOUCHEMAL, Paris Cedex 05 (FR); Jean-Maurice MALLET, Paris (FR)

(21) Appl. No.: 19/474,798

(22) PCT Filed: Apr. 19, 2024
(86) PCT No.: PCT/EP2024/060785

§ 371 Date: Oct. 13, 2025

(30) Foreign Application Priority Data
Apr. 19, 2023 (FR) FR2303940

(51) Int. Cl. A61K 8/60; A61K 8/73; A61K 9/16; A61Q 19/00

(52) U.S. Cl. CPC A61K 8/602; A61K 8/738; A61K 9/1623; A61K 9/1652; A61Q 19/00

(57) ABSTRACT

The present invention relates to particles comprising at least one monosaccharide modified with a hydrophobic chain, at least one cyclodextrin, and optionally a molecule of therapeutic or cosmetic interest, to the preparation method thereof, to a pharmaceutical or cosmetic composition comprising same, and to the use thereof for the sustained release of molecules of cosmetic or therapeutic interest. The invention also relates to said modified monosaccharide.

### TECHNICAL FIELD OF THE INVENTION

The present invention relates to particles comprising at least one monosaccharide modified with a hydrophobic chain, at least one cyclodextrin, and optionally a molecule of therapeutic or cosmetic interest, to the preparation method thereof, to a pharmaceutical or cosmetic composition comprising same, and to the use thereof for the sustained release of molecules of cosmetic or therapeutic interest. The invention also relates to said modified monosaccharide.

### STATE OF THE ART

The encapsulation of active ingredients in particles has been developed with a view to allowing better control of the release of active ingredients and/or to isolate them from the environment in which they are found in order to avoid any degradation or unwanted interaction.

Microparticles (the size of which is comprised between 1 and 1000 μm) and nanoparticles (the size of which is less than 1 μm) can be obtained by three main classes of methods: (1) physicochemical methods based on variations in solubility of the materials used for encapsulation under the effect of variations in temperature or pH, (2) mechanical methods such as extrusion for example and (3) chemical methods during which the synthesis of the membrane that makes up the particle, generally based on polymer, and the encapsulation of the active substances take place simultaneously.

Microencapsulation and nanoencapsulation techniques such as nanoprecipitation, solvent emulsion-evaporation, salting-out or solvent emulsion-diffusion generally use surfactants and organic solvents (Salatin et al. 2018).

Regarding mechanical methods, they often require complex equipment. In addition, the increased temperature represents a significant energy loss and can lead to partial or complete denaturation of heat-sensitive active ingredients.

Finally, among category (3) of methods, polymerization or polycondensation methods, if they have the advantage of not using solvents, have the disadvantage of using extremely reactive products capable of reacting with the substances to be encapsulated. For example, in the case of emulsion polymerization of alkylcyanoacrylates, the polymerization reagents can react chemically with the active ingredient to be encapsulated. This is the cause of a partial or total loss of the effectiveness of the formulations. This method also uses extreme variations in pH, which is also the cause of the degradation of part or all of the active ingredient (Mirchandani et al. 2021).

Thus, the methods for encapsulating molecules of interest pose several problems.

The main problem remains the toxicity due to the solvents, surfactants, or reagents used during encapsulation, as well as the extreme variation in pH during the preparation method. In some cases, heating or strong agitation is also necessary, which results in a loss of energy and time. In addition, the molecules to be encapsulated may undergo degradation due to the solvents, surfactants, and reagents used, pH variations, or else the strong agitation or heating required for the manufacture of the particles, leading to therapeutic ineffectiveness of the resulting particles.

Encapsulation methods, particularly by interfacial polycondensation or by solvent emulsion-diffusion, are lengthy because they generally involve several steps, with additional purification steps to remove excess reagents, solvents or surfactants often being necessary.

Finally, when encapsulation is possible, the encapsulation yield is generally low and the release of the encapsulated active substance is most often uncontrolled, with a “burst release” effect being conventionally observed.

Therefore, there remains a need for a method for encapsulating molecules of therapeutic or cosmetic interest that is simple, does not involve the use of toxic ingredients, while limiting the degradation of the encapsulated active substances.

Cyclodextrins (CDs) are cyclic oligosaccharides composed of α-(1,4)-linked glucopyranose chains. For example, alpha-cyclodextrin (α-CD) is composed of six glucose units in chair conformation linked by α-1,4 glycosidic bonds (FIG. 1). CDs appear as a conical frustum. The apolar cavity of CDs is lined by hydrogen atoms linked to the C3 and C5 carbon atoms and by oxygen atoms forming ether bonds, while hydroxyl groups are found on the outside (Del Valle 2004). The amphiphilic nature of CDs, both hydrophilic on the outside and hydrophobic on the inside, allows them to include hydrophobic molecules in their cavity to form inclusion complexes (Diaz-Salmeron et al. 2018). Furthermore, the cavity contains water molecules whose presence is energetically unfavorable (polar-apolar interaction), which allows substitution by guest molecules of less polarity than water (Rekharsky et al. 1998).

Studies have highlighted the possibility of forming self-assembled systems such as micelles, nanoparticles or vesicles through non-covalent interactions between α-CD and a hydrophobic guest molecule. Such systems are similar to colloids intended to vectorize active substances in the pharmaceutical field, to protect vitamins in the food industry or to stabilize/protect active substances against heat or light or to control the release of active substances in the cosmetic field (Crini et al. 2021).

Similarly, multiple α-CD molecules can thread along another linear molecule ‘axis’ forming inclusion complexes, the resulting assemblies being called polyrotaxanes. Spherical polyrotaxane nanoparticles were thus obtained after mixing α-CD with PEG modified by cinnamic acid at each end of the PEG chain (Zhang et al. 2014). Nanorods and microrods were also obtained by mixing tetradecane with α-CD solution (Mathapa et al. 2013).

Finally, nano- and micro-platelets could be obtained by the hierarchical assembly in water of α-CD and polysaccharides hydrophobically modified by C16-C18 alkyl chains. The formation of structured and cohesive platelets is induced by the interaction of the alkyl chains grafted onto the polysaccharides with the α-CD molecules and the self-organization of the inclusion complexes with each other allows the formation of a multilayer structure (Ahmed et al. 2018). International application WO 2013/150193 describes in particular the formation of nanoplatelets resulting from the interaction between a chitosan modified with an alkyl chain and α-CD. However, the nanoplatelet shapes make it difficult to encapsulate an active ingredient as well as its controlled release. In addition, the compositions obtained do not have a rheofluidifying character which is of interest in particular for topical application.

### SUMMARY OF THE INVENTION

Surprisingly, the Applicant has discovered that particles comprising at least one hydrophobic-chain-modified monosaccharide and a cyclodextrin allow to effectively encapsulate hydrophilic molecules of interest while having a simple and versatile preparation method. In addition, the compositions obtained have good shear-thinning properties which are interesting for topical application.

A first object of the invention therefore relates to particles comprising:
at least one compound having the following formula:

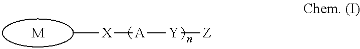

in which
   n represents 0 or 1;
   M represents a residue of a monosaccharide (for example aldohexose or aldopentose), preferably of an aldohexose, of which an OH group, preferably the OH group in the anomeric position, is replaced by the group —X-(A-Y)ₙ—Z and of which one or more —OH groups are optionally replaced by a group —OSO₃H and/or of which a group —CH₂—OH is optionally replaced by a group —COOH;
   X represents —O—, —S—, —NR₁—, —OC(O)—, —SC(O)—, —N(R₁)C(O)—, —OC(S)—, —SC(S)—, or —N(R₁)C(S)—, the first atom of the groups being linked to M;
   A represents a linker,
   the linker being a (C₁-C₂₀)alkyl chain, in particular (C₁-C₁₅)alkyl, in particular (C₁-C₁₀)alkyl, and preferably (C₁-C₈)alkyl, optionally interrupted one or more times by one or more moieties
   selected from the group consisting of —O—, —S—, —NR₂—, —C(O)—, and —C(S)—, and/or optionally substituted with one or more groups selected from the group consisting of a halogen atom, —NO₂, —CN, —OR₃, —SR₄, and —NR₅R₆;
   Y represents —O—, —S—, —NR₇—, —C(O)—, —C(S)—, —OC(O)—, —C(O)O—, —SC(O)—, —C(O)S—, —N(R₇)C(O)—, —C(O)N(R₇)—, —OC(S)—, —C(S)O—, —SC(S)—, —C(S)S—, —N(R₇)C(S)—, or —C(S)N(R₇)—;
   R₁ to R₇ represent, independently of each other, a hydrogen atom or a (C₁-C₆)alkyl group (for example (C₁-C₄)alkyl); and
   Z represents a saturated or unsaturated hydrocarbon chain, in particular saturated, linear or branched, preferably linear, comprising from 4 to 40 carbon atoms, in particular 6 to 28 carbon atoms, preferably 12 to 24 carbon atoms; and
at least one cyclodextrin, preferably α-cyclodextrin.

According to a particular embodiment of the invention, the particles further comprise at least one molecule of therapeutic or cosmetic interest.

A second object of the invention relates to a method for preparing the particles according to the invention comprising mixing:
at least one compound of formula Chem. (I) as defined above;
at least one cyclodextrin, preferably α-cyclodextrin; and
optionally a molecule of therapeutic or cosmetic interest.

A third object of the invention relates to a compound having the following formula:

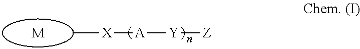

in which
n represents 0 or 1;
M represents a residue of a monosaccharide (for example aldohexose or aldopentose), preferably of an aldohexose, of which an OH group, preferably the OH group in the anomeric position, is replaced by the group —X-(A-Y)ₙ—Z and of which one or more —OH groups are optionally replaced by a group —OSO₃H and/or of which a group —CH₂—OH is optionally replaced by a group —COOH;
X represents —O—, —S—, —NR₁—, —OC(O)—, —SC(O)—, —N(R₁)C(O)—, —OC(S)—, —SC(S)—, or —N(R₁)C(S)—, the first atom of the groups being linked to M;
A represents a linker,
the linker being a (C₁-C₂₀)alkyl chain, in particular (C₁-C₁₅)alkyl, in particular (C₁-C₁₀)alkyl, and preferably (C₁-C₈)alkyl,
optionally interrupted one or more times by one or more moieties selected from the group consisting of —O—, —S—, —NR₂—, —C(O)—, and —C(S)—, and/or optionally substituted with one or more groups selected from the group consisting of a halogen atom, —NO₂, —CN, —OR₃, —SR₄, and —NR₅R₆;
Y represents —O—, —S—, —NR₇—, —C(O)—, —C(S)—, —OC(O)—, —C(O)O—, —SC(O)—, —C(O)S—, —N(R₇)C(O)—, —C(O)N(R₇)—, —OC(S)—, —C(S)O—, —SC(S)—, —C(S)S—, —N(R₇)C(S)—, or —C(S)N(R₇);
R₁ to R₇ represent, independently of each other, a hydrogen atom or a (C₁-C₆)alkyl group; and
Z represents a saturated or unsaturated hydrocarbon chain, in particular saturated, linear or branched, preferably linear, comprising from 4 to 40 carbon atoms, preferably 6 to 24 carbon atoms.

A fourth object of the invention relates to a cosmetic or pharmaceutical composition comprising particles according to the invention and at least one cosmetically or pharmaceutically acceptable excipient.

A fifth object of the invention relates to the use of particles according to the invention comprising at least one molecule of cosmetic interest for the sustained release of the molecule of cosmetic interest.

The invention also relates to a method for the sustained release of a molecule of cosmetic interest comprising the administration, for example topically, to a person in need thereof of an effective amount of particles according to the invention comprising the molecule of cosmetic interest.

A sixth object of the invention relates to particles according to the invention comprising at least one molecule of therapeutic interest, for their use as a medicament, preferably as a prolonged-release medicament.

The invention also relates to the use of particles according to the invention comprising at least one molecule of therapeutic interest, for the preparation of a prolonged-release medicament.

The invention also relates to a method for the sustained release of a molecule of therapeutic interest comprising the administration, for example topically, to a person in need thereof of an effective amount of particles according to the invention comprising the molecule of therapeutic interest.

### DETAILED DESCRIPTION OF THE INVENTION

For the purposes of the present invention, the various embodiments presented throughout the description may be used alone or in combination with each other, without limitation of combination.

### Definitions

For the purposes of the present invention, the term “monosaccharide” means a compound of formula H—(CHOH)ₓ—CO—(CHOH)_{y}—H with x and y representing, independently of each other, an integer ranging from 0 to 5 provided that 2≤x+y, and in particular x+y=2, 3, 4, 5, 6, 7, 8 or 9, and preferably x+y=4 or 5, the monosaccharide possibly being in a linear form represented by the abovementioned formula or possibly being in a cyclized form by reaction of the CO (aldehyde or ketone) function with one of the OH groups to form a hemiacetal or hemiketal group. Preferably, the monosaccharide is in cyclized form. The monosaccharide may be an aldose which carries an aldehyde function (when x or y is 0) or a ketose which carries a ketone function (when neither x nor y is 0). It will preferably be an aldose. Preferably, the monosaccharide is a hexose (comprising 6 carbon atoms), that is to say x+y=5, and in particular an aldohexose. The monosaccharide may also be a pentose (comprising 5 carbon atoms), that is to say x+y=4, and in particular an aldopentose. The monosaccharide may be of the dextrorotatory (D) or levorotatory (L) type, preferably of the dextrorotatory (D) type. The monosaccharide may in particular be allose, altrose, glucose, mannose, gulose, idose, galactose, talose, ribose, arabinose, xylose, or lyxose. Preferably, it will be mannose, galactose, xylose or glucose, and in particular D-mannose, D-galactose, D-xylose or D-glucose. According to one embodiment, it will be mannose, galactose or glucose, and in particular D-mannose, D-galactose, or D-glucose.

In the context of the present invention, one or more —OH groups of the monosaccharide are optionally replaced by an —OSO₃H group and/or a —CH₂—OH group of the monosaccharide is optionally replaced by a group —COOH.

For the purposes of the present invention, the term “monosaccharide residue” means a monosaccharide as defined above from which an OH group has been removed and which is linked to the rest of the molecule by the carbon atom originally bearing the removed OH group.

For the purposes of the present invention, the term “alkyl” group means a saturated, linear or branched hydrocarbon chain.

The term “(C₁-Cₓ)alkyl” means an alkyl group as defined above, including from 1 to x carbon atoms. Thus, a (C₁-C₆)alkyl group comprises 1 to 6 carbon atoms and may be, for example, a methyl, ethyl, isopropyl, tert-butyl, pentyl, or hexyl group. Similarly, a (C₁-C₂₀)alkyl group comprises 1 to 20 carbon atoms and may be, for example, a methyl, ethyl, isopropyl, tert-butyl, pentyl, hexyl, or decyl group.

For the purposes of the present invention, the term “(C₂-C₆)alkenyl” group means a linear or branched hydrocarbon chain comprising at least one double bond and comprising 2 to 6 carbon atoms. For example, ethenyl or allyl groups may be mentioned.

For the purposes of the present invention, the term “aryl” means an aromatic hydrocarbon group, preferably comprising from 6 to 10 carbon atoms, and comprising one or more fused rings, such as, for example, a phenyl or naphthyl group. Advantageously, this is phenyl.

For the purposes of the present invention, the term “aryl-(C₁-C₆)alkyl” means an aryl group as defined above, linked to the rest of the molecule via a (C₁-C₆)alkyl chain as defined above. By way of example, the benzyl group may be mentioned.

For the purposes of the present invention, the term “(C₁-C₆)alkyl-aryl” means a (C₁-C₆)alkyl group as defined above, linked to the rest of the molecule via an aryl group as defined above. By way of example, mention may be made of the tolyl group (CH₃Ph).

For the purposes of the present invention, the term “halogen atom” means fluorine, chlorine, bromine and iodine atoms.

For the purposes of the present invention, the term “cyclodextrin” means a cyclic oligosaccharide composed of glucopyranose chains linked in α-(1,4). Preferably, the cyclodextrin comprises 6, 7 or 8, in particular 6, glucopyranose chains.

As used herein, “cosmetically or pharmaceutically acceptable” means that which is useful in the preparation of a cosmetic or pharmaceutical composition which is generally safe, non-toxic and neither biologically nor otherwise undesirable and which is acceptable for cosmetic or pharmaceutical use, particularly human pharmaceutical use.

For the purposes of the present invention, “room temperature” means a temperature comprised between 8 and 40° C., preferably between 15 and 30° C., in particular between 2° and 25° C.

For the purposes of the present invention, the term “protecting group” means a group which selectively blocks a reactive site in a multifunctional compound such that a chemical reaction can be carried out selectively at another unprotected reactive site in the meaning conventionally associated therewith in synthetic chemistry.

For the purposes of the present invention, the term “O-protecting group” means any substituent that protects the hydroxyl group, that is to say a reactive oxygen atom, against undesirable reactions such as the O-protecting groups described in “Greene's Protective Groups In Organic Synthesis”, 4^{th} edition, 2007, John Wiley & Sons, Hoboken, New Jersey. A hydroxyl group protected by an O-protecting group may be, for example, an ether, an ester, a carbonate, an acetal and the like. In particular, the O-protecting groups comprise a (C₁-C₆)alkyl group optionally substituted by one or more (in particular 1 to 3) halogen atoms (such as chlorine atoms), such as methyl, ethyl, tert-butyl and 2,2,2-trichloroethyl groups; an aryl-(C₁-C₆)alkyl group, such as benzyl, the aryl ring being optionally substituted by one or more methoxy groups, such as benzyl (Bn) and p-methoxybenzyl (PMB) groups; a trityl group of formula —CAr₁Ar₂Ar₃, such as triphenylmethyl (also called trityl—Tr), (4-methoxyphenyl) diphenylmethyl (also called methoxytrityl—NMT) and bis-(4-methoxyphenyl) phenylmethyl (also called dimethoxytrityl—DMT); a substituted methyl group of formula —CH₂OR_{GP2} or —CH₂SR_{GP2} (in particular —CH₂OR_{GP2}), such as methoxymethyl (MOM), benzyloxymethyl, 2-methoxyethoxymethyl (MEM), 2-(trimethylsilyl) ethoxymethyl and methylthiomethyl; a substituted ethyl group of formula —CH₂CH₂OR_{GP2} or —CH₂CH₂SR_{GP2} (in particular —CH₂CH₂OR_{GP2}), such as ethoxyethyl (EE); a silyl group of formula —SiR_{GP3}R_{GP4}R_{GP5}, such as trimethylsilyl (TMS), triethylsilyl (TES), t-butyldimethylsilyl (TBS or TBDMS) and t-butyldiphenylsilyl (TBDPS) groups; a carbonyl group of formula —CO—R_{GP6}, such as acetyl (Ac), pivaloyl (Piv or Pv) and benzoyl (Bz) groups, or of formula —CO₂—R_{GP7}, such as allyloxycarbonyl (Alloc) and 9-fluorenylmethyloxycarbonyl (Fmoc) groups; or a tetrahydropyranyl group

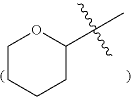 [SMILES: *C1CCCCO1]

(THP) or tetrahydrofuranyl

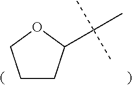

with Ar₁, Ar₂ and Ar₃ representing, independently of each other, an aryl, such as phenyl, optionally substituted by one or more methoxy groups; R_{GP2} representing a (C₁-C₆)alkyl group (such as methyl or ethyl) optionally substituted by an aryl group (such as phenyl), (C₁-C₆)alkoxy (such as methoxy) or trialkylsilyl (such as SiMe₃); R_{GP3}, R_{GP4} and R_{GP5} representing, independently of each other, a (C₁-C₆)alkyl or aryl group (such as phenyl); and R_{GP6} and R_{GP7} representing, independently of each other, a (C₁-C₆)alkyl, (C₂-C₆)alkenyl, aryl, aryl-(C₁-C₆)alkyl or 9-fluorenylmethyl group.

In particular, it is an acetyl group. It could also be a benzoyl group.

For the purposes of the present invention, the term “leaving group” means a chemical group which can be easily displaced by a nucleophile during a nucleophilic substitution reaction, the nucleophile being, for example, an alcohol, thiol or amine function. Such a leaving group may more particularly be a halogen atom such as a chlorine or bromine atom, a sulfonate, an N-succinimidyloxy group, a 4-nitro-phenyloxy group, a pentafluorophenoxy group or an N-benzotriazoloxy group. The sulfonate may in particular be a group —OSO₂—R_{LG} with R_{LG} representing a (C₁-C₆)alkyl, aryl, aryl-(C₁-C₆)alkyl or (C₁-C₆)alkyl-aryl group, said group being optionally substituted by one or more halogen atoms such as fluorine atoms. The sulfonate can be in particular a mesylate (—OS(O₂)—CH₃), a triflate (—OS(O)₂—CF₃) or else a tosylate (—OS(O)₂-(p-Me-C₆H₄)).

For the purposes of the present invention, the term “activated form” of the COOH or CSOH group means a COOH or CSOH group in which the OH moiety of the COOH or CSOH function has been replaced by an activated leaving group (LG) allowing the coupling of the COOH or CSOH group in an activated form with a hydroxyl (OH), thiol (SH) or amino (NH) function by formation of a (thio)ester (C(OO, C(O)—S, C(S)—O, C(S)—S) or (thio)amide (C(O)—N, C(S)—N) bond and release of the LG-H compound. The activated forms may be activated (thio)esters, activated (thio)amides, anhydrides or (thio)acyl halides such as (thio)acyl chlorides. The activated (thio)esters include derivatives formed by the reaction of the COOH or CSOH group with N-hydroxybenzotriazole or N-hydroxysuccinimide.

### Compound of Formula Chem. (I)

The present invention relates to a compound having the following formula:

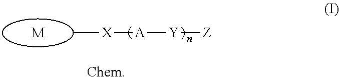

M, X, A, Y, n and Z being as defined above, and
at least one cyclodextrin, preferably α-cyclodextrin.

M may represent an aldohexose or aldopentose residue, in particular mannose, galactose, xylose or glucose, and more particularly D-mannose, D-galactose, D-xylose or D-glucose, one or more —OH groups of which are optionally replaced by a group —OSO₃H and/or one group —CH₂—OH of which is optionally replaced by a group —COOH.

Preferably, M represents an aldohexose residue, in particular mannose, galactose or glucose, and more particularly D-mannose, D-galactose or D-glucose, one or more —OH groups of which are optionally replaced by a group —OSO₃H and/or one group —CH₂—OH of which is optionally replaced by a group —COOH. Preferably, M represents a mannose residue, more particularly D-mannose, one or more —OH groups of which are optionally replaced by a group —OSO₃H and/or one group —CH₂—OH of which is optionally replaced by a group —COOH. Preferably, M represents a mannose residue, in particular D-mannose.

Advantageously, X represents —O—, —NR₁—, —OC(O)—, or —N(R₁)C(O)—; in particular —O— or —N(R₁)C(O)—; in particular —O— or —NHCO—, the first atom of the groups being linked to M. Preferably, X represents —O—.

According to a first embodiment of the invention, n=0 and the moiety A-Y is absent.

According to a second preferred embodiment of the invention, n is equal to 1 and the moiety A-Y is present.

In this embodiment, A advantageously represents a (C₁-C₂₀)alkyl chain, in particular (C₁-C₁₅)alkyl, in particular (C₁-C₁₀)alkyl, and preferably (C₁-C₈)alkyl, optionally interrupted one or more times by one or more moieties selected from the group consisting of —O—, —NR₂—, and —C(O)—, and/or optionally substituted with one or more groups selected from the group consisting of a halogen atom, —OR₃, and —NR₅R₆. In particular, A advantageously represents a (C₁-C₂₀)alkyl chain, in particular (C₁-C₁₅)alkyl, in particular (C₁-C₁₀)alkyl, and preferably (C₁-C₈)alkyl, optionally interrupted one or more times by one or more —O— moieties and/or optionally substituted with one or more —OR₃ groups, such as OH. Preferably, A represents a (C₁-C₂₀)alkyl chain, in particular (C₁-C₁₅)alkyl, in particular (C₁-C₁₀)alkyl, and preferably (C₁-C₈)alkyl, such as a —(CH₂)ₘ— chain with m representing 1, 2, 3, 4, 5, 6, 7 or 8, in particular 2 or 3, in particular 3. A may also represent a chain —CH₂CH₂—(O—CH₂CH₂)ₚ— with p=1, 2, 3, 4 or 5, in particular 1.

Likewise, in this embodiment, Y advantageously represents —O—, —NR₇—, —C(O)—, —OC(O)—, —C(O)O—, —N(R₇)C(O)—, or —C(O)N(R₇)—; in particular —OC(O)—, —C(O)O—, —N(R₇)C(O)— or —C(O)N(R₇)—; more particularly —N(R₇)C(O)— or —C(O)N(R₇)— (for example —NHC(O) or —C(O)NH—); preferably —N(R₇)C(O)— such as —NHC(O)—, the first atom of the groups being linked to A.

According to a preferred embodiment, n is equal to 1, A represents a —(CH₂)ₘ— chain with m representing 1, 2, 3, 4, 5, 6, 7 or 8, in particular 2 or 3, in particular 3, and Y represents —OC(O)—, —C(O)O—, —N(R₇)C(O)— or —C(O)N(R₇)—, in particular —N(R₇)C(O)— or —C(O)N(R₇)— (for example —NHC(O) or —C(O)NH—), preferably —N(R₇)C(O)— such as —NHC(O)—, the first atom of the groups being linked to A.

Preferably, Z represents a linear, saturated or unsaturated hydrocarbon chain of 4 to 40, in particular 6 to 28, preferably 12 to 24 carbon atoms, typically 15 or 17 carbon atoms.

According to a preferred embodiment, n is equal to 1, X represents —O— or —N(R₁)C(O)— such as —NHCO— preferably —O—, A represents a chain —(CH₂)ₘ— with m representing 1, 2, 3, 4, 5, 6, 7 or 8, in particular 2 or 3, in particular 3, and Y represents —N(R₇)C(O)— or —C(O)N(R₇)— (for example —NHC(O) or —C(O)NH—), preferably —N(R₇)C(O)— such as —NHC(O)—, the first atom of the groups being linked to A.

According to one embodiment, n is equal to 1, X represents —O— or —N(R₁)C(O)— such as —NHCO— preferably —O—, A represents a chain —(CH₂)ₘ— substituted by an OH group with m representing 1, 2, 3, 4, 5, 6, 7 or 8, in particular 2 or 3, in particular 3, and Y represents —N(R₇)C(O)— or —C(O)N(R₇)— (for example —NHC(O) or —C(O)NH—), preferably —N(R₇)C(O)— such as —NHC(O)—, the first atom of the groups being linked to A.

According to another embodiment, n is equal to 1, X represents —O— or —N(R₁)C(O)— such as —NHCO— preferably —O—, A represents —CH₂CH₂—(O—CH₂CH₂)ₚ— with p=1, 2, 3, 4 or 5, in particular 1 and Y represents —N(R₇)C(O)—, N(R₇) being linked to A.

According to another embodiment, n is equal to 0, X represents —O— or —N(R₁)C(O)—, preferably —N(R₁)C(O)— such as —NHCO—, the first atom of the groups being linked to M.

According to a preferred embodiment:
M represents a residue of mannose, galactose, xylose or glucose, and more particularly of D-mannose, D-galactose, D-xylose or D-glucose, one or more —OH groups of which are optionally replaced by a group —OSO₃H and/or one group —CH₂—OH of which is optionally replaced by a group —COOH;
n is equal to 1;
X represents —O—;
A represents a —(CH₂)ₘ— chain with m representing 1, 2, 3, 4, 5, 6, 7 or 8;
Y represents —OC(O)—, —C(O)O—, —N(R₇)C(O)— or —C(O)N(R₇)— with R₇ as defined above, preferably Y represents —NHC(O)— or —C(O)NH—; and
Z represents a saturated or unsaturated hydrocarbon chain, in particular saturated, linear or branched, preferably linear, comprising from 12 to 24 carbon atoms, typically 15 or 17 carbon atoms.

Preferably, the molecule of formula Chem. (I) corresponds to the following formula Chem. (II) (this compound is called ML in the experimental part, including the figures):

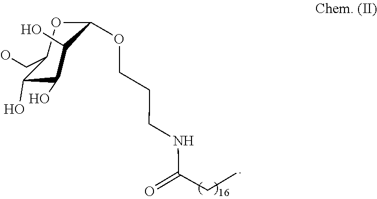

The compound ML can also be represented by the following formula:

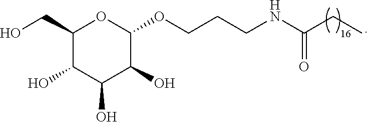

According to a preferred embodiment, the molecule of formula Chem. (I) is selected from the group consisting of

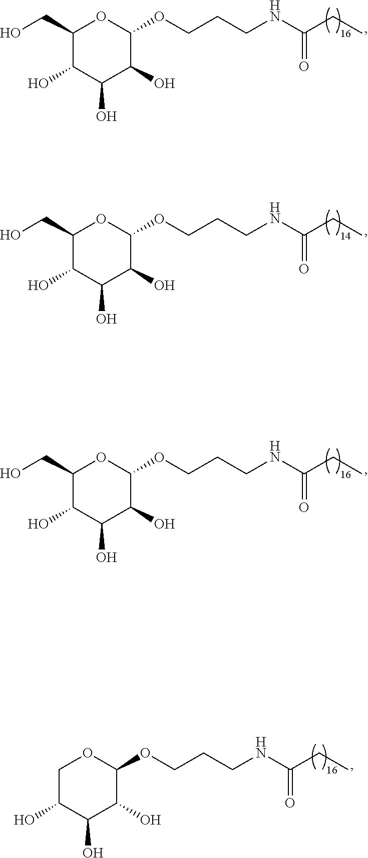

and mixtures thereof.

### Method for Preparing the Compound of Formula Chem. (I)

The compound of formula Chem. (I) may be prepared by a preparation method comprising a coupling reaction at X and a coupling reaction at Y, when Y is present, the coupling reaction depending on the nature of X and Y.

The preparation method described below is suitable for preparing all kinds of compounds of formula Chem. (I). For example, this preparation method allows to prepare compounds of formula Chem. (I) both when M is an aldohexose, such as mannose, galactose or glucose, and when M is an aldopentose such as xylose.

When n=0, the preparation method advantageously comprises a step of coupling M₁-X₁ with X₂—Z, for which:
M₁ representing a group M as defined above, optionally in a protected form, and preferably in which the —OH groups are in a protected form and therefore substituted by an 0-protecting group such as an acetyl or a benzoyl, in particular an acetyl;
Z is as defined above;
X₁ and X₂ each represent a reactive chemical group capable of reacting together to form a group X as defined above.

According to a first embodiment, the group X₁ is a group —OH, —SH or —NR₁H and the group X₂ is a leaving group, in particular as defined above, or a group —COOH or —CSOH optionally in an activated form.

According to a second embodiment, the group X₁ is a leaving group, in particular as defined above and the group X₂ is a group —OH, —SH or —NR₁H.

In this embodiment, when the group X₁ is located in the anomeric position of the monosaccharide M, X₁ may in particular be an —OCO—R₈ group with R₈ representing a (C₁-C₆)alkyl group such as methyl. Preferably, the other OH groups of the monosaccharide M will be in a protected form. The coupling reaction of this group X₁ with X₂ representing a —OH, —SH or —NR₁H group may then be carried out in the presence of a Lewis acid.

Lewis acids are well known to the person skilled in the art. They can be selected from boron trihalides such as boron trifluoride (for example BF₃·OEt₂), phosphorus pentahalides, arsenic pentahalides and antimony pentahalides.

The coupling step between X₁ and X₂ allowing to form the moiety X is well known to the person skilled in the art.

When M₁ represents a group M in a protected form, an additional deprotection step will be necessary to obtain the desired compound Chem. I.

When n=1, according to a first alternative, the preparation method may comprise the coupling of M₁-X₁ with X₂-A-Y—Z, for which M₁, X₁, X₂, A, Y and Z are as defined above.

The molecule X₂-A-Y—Z can be prepared by coupling between X₂′-A-Y₁ and Y₂—Z, for which A and Z are as defined above and X₂′ represents a group X₂ as defined above, optionally in a protected or inactivated form. Y₁ and Y₂ each represent a reactive chemical group capable of reacting together to form a group Y as defined above. The chemical groups Y₁ and Y₂ can be chosen according to Table 1 below. The step of coupling between Y₁ and Y₂ to form the moiety Y is well known to the person skilled in the art.

TABLE 1

| | 2^{nd} reactive group |
|---|---|
| 1^{st} reactive | (Y₁ or Y₂) |
| group (Y₁ | able to react with |
| or Y₂) | the 1^{s}t reactive group |
| OH, SH or NHR₇ | COOH or CSOH or an activated |
| | form such as C(O)Cl |
| OH, SH or NHR₇ | Leaving group such as a halogen |
| | atom or a sulfonate |
| C(O)Hal or C(S) Hal | MgHal |

Hal represents a halogen atom such as Br or Cl, R₇ is as defined above.

When n=1, according to a second alternative, the preparation method may comprise the coupling of M₁-X-A-Y₁ with Y₂—Z, for which M₁, X, A, Y₁, Y₂ and Z are as defined above.

The molecule M₁-X-A-Y₁ can be prepared by coupling between M₁-X₁ and X₂-AY₁′, for which M₁, X₁, X₂, A and Z are as defined above, and Y₁′ represents a group Y₁ as defined above optionally in a protected or inactive form.

For the purposes of the present invention, “inactive form” means that the group Y₁′ is not capable of coupling with Y₂ to form a moiety Y. When Y₁′ represents a group Y₁ in a protected or inactive form, it will be deprotected or activated so as to obtain the group Y₁ before carrying out the coupling reaction. The deprotection or activation reaction can be carried out in situ, that is to say that the deprotection or activation reaction can be carried out during the same reaction step as the coupling. The deprotection or activation reaction can also be carried out in one or two reaction steps.

For example, when Y₁ represents —NH₂, then Y₁′ can be a halogen atom or an N₃ group. Halogen atoms (for example Br or Cl) can be easily substituted by an azide, this azide can then be reduced to an amine group in the presence of a phosphorus(III) derivative such as triphenylphosphine (Staudinger reduction). Thus, for example, Y₁′ can represent a bromine or chlorine atom or an N₃ group when Y₁ represents an NH₂ group.

The compound Chem. (I) thus obtained can be separated from the reaction medium by methods well known to the person skilled in the art, such as for example by extraction, evaporation of the solvent or by precipitation and filtration.

The compound may also be purified if necessary by techniques well known to the person skilled in the art, such as by recrystallization if the compound is crystalline, by distillation, by column chromatography on silica gel or by high performance liquid chromatography (HPLC).

### Particles

The present invention also relates to particles comprising:
at least one compound of formula Chem. (I), and
at least one cyclodextrin, preferably α-cyclodextrin.

The compound of formula Chem. (I) is as defined above.

In a preferred embodiment, the particles according to the invention comprise:
at least one compound selected from the group consisting of group

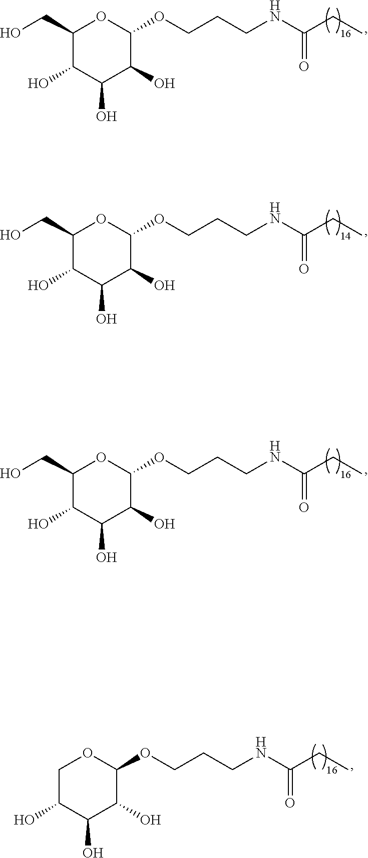

and
at least one α-cyclodextrin.

In a particularly preferred embodiment, the particles according to the invention comprise:
at least one compound having the following formula:

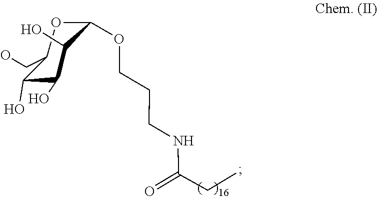

and
at least one α-cyclodextrin.

The molar ratio between the compound Chem. (I) and the cyclodextrin is advantageously from 1 to 20, preferably from 5 to 10.

Advantageously, the particles according to the invention may have a hydrodynamic diameter ranging from 10 nm to 10 μm, preferably from 40 nm to 4 μm, more preferably from 200 nm to 2 μm and/or a number average diameter D_{TEM} ranging from 10 nm to 10 μm, in particular from 20 nm to 1 μm, in particular from 50 nm to 300 nm.

The hydrodynamic diameter can be determined by dynamic light scattering (DLS), in particular from a suspension of the particles in water, more particularly according to the method described in the experimental part. The particles according to the invention suspended in a solvent such as water can form aggregates and appear in the form of a nanogel. The hydrodynamic diameter allows to measure the size of these aggregates.

The number-average diameter D_{TEM} can be determined by transmission electron microscopy (TEM), which provides a distribution of the number diameters of the particles, the average of which is called D_{TEM}. This diameter can be measured more specifically using the method described in the experimental section. In this case, the diameter measured is that of the particles taken individually, not of the aggregates.

The particles according to the invention may further comprise at least one molecule of therapeutic or cosmetic interest, whether hydrophilic or lipophilic.

The molecule of therapeutic interest may be a hydrophilic or lipophilic substance belonging to classes I and III of the Biopharmaceutical Classification System, namely: antibiotics, peptides such as antimicrobial peptides (for example temporins), antiviral agents, antiparasitic agents (for example metronidazole or fluconazole), antihistamines (for example cimetidine), analgesics (for example paracetamol), vitamins (for example ascorbic acid) or anti-inflammatory agents (for example ibuprofen).

The molecule of cosmetic interest may be selected from amino acids, salicylic acid, betaine, caffeine, peptides used in cosmetology (for example tripeptide-1, palmitoyl tripeptide-1, palmitoyl tetrapeptide-7, palmitoyl hexapeptide-12, myristoyl hexapeptide-16, myristoyl pentapeptide-17, hexanoyl dipeptide-3 norleucine acetate, azelaoyl bis-dipeptide-10 and mixtures thereof) and mixtures thereof.

Antibiotics include: 3-lactams such as penicillins (for example, piperacillin), carbapenems (for example, meropenem), monobactams (for example, aztreonam), cephalosporins (for example, ceftriaxone), glycopeptides (for example, teicoplanin and vancomycin), aminoglycosides (for example, gentamicin and amikacin), oxazolidinones (for example, linezolid), and mixtures thereof.

Antiviral agents include: abacavir, acyclovir (aciclovir), adefovir, amantadine, ampligen, amprenavir (agenerase), umifenovir (arbidol), atazanavir, atripla (efavirenz/emtricitabine/tenofovir), baloxavir marboxil (Xofluza), biktarvy (Bictegravir/emtricitabine/tenofovir alafenamide), boceprevir, bulevirtide, lecidofovir, cobicistat (Tybost), combivir (Lamivudine/Zidovudine), daclatasvir (Daklinza), darunavir, delavirdine, descovy (Emtricitabine/tenofovir alafenamide), didanosine, docosanol, dolutegravir, doravirine (Pifeltro), edoxudine, efavirenz, elvitegravir, emtricitabine, enfuvirtide, ensitrelvir, entecavir, etravirine (Intelence), famciclovir, fomivirsen, fosamprenavir, foscarnet, ganciclovir (Cytovene), ibacitabine, ibalizumab (Trogarzo), idoxuridine, imiquimod, inosine pranobex, indinavir, lamivudine, letermovir (Prevymis), lopinavir, loviride, maraviroc, methisazone, moroxydine, nelfinavir, nirmatrelvir/ritonavir (Paxlovid), nevirapine, nitazoxanide, norvir, oseltamivir (Tamiflu), penciclovir, peramivir (Rapivab), pleconaril, podophyllotoxin, raltegravir, remdesivir, ribavirin, rilpivirine (Edurant), rimantadine, ritonavir, saquinavir, simeprevir (Olysio), sofosbuvir, stavudine, taribavirin (Viramidine), telaprevir, telbivudine (Tyzeka), tenofovir alafenamide, tenofovir disoproxil, tipranavir, trifluridine, trizivir, tromantadine, truvada, umifenovir, valaciclovir (Valtrex), valganciclovir (Valcyte), vicriviroc, vidarabine, zalcitabine, zanamivir (Relenza), zidovudine, and mixtures thereof.

In particular, the molecule of therapeutic or cosmetic interest may be selected from the group consisting of acetylsalicylic acid, acyclovir, amitriptyline hydrochloride, amodiaquine hydrochloride, amoxicillin trihydrate, atenolol, bisoprolol fumarate, chloroquine hydrochloride or phosphate or sulfate, cimetidine, ciprofloxacin hydrochloride, codeine phosphate, sodium or potassium diclofenac, doxycycline hyclate, efavirenz, enalapril maleate, ethambutol dihydrochloride, fluconazole, folic acid, furosemide, ibuprofen, ketoprofen, isoniazid, lamivudine, levetiracetam, levofloxacin, metoclopramide hydrochloride, metronidazole, nifedipine, piroxicam, prednisolone, prednisone, primaquine diphosphate, proguanil hydrochloride, propranolol hydrochloride, pyrazinamide, quinidine sulfate, quinine sulfate, ranitidine hydrochloride, ribavirin, rifampicin, stavudine, verapamil hydrochloride, zidovudine, allopurinol, amiloride hydrochloride, amitriptyline hydrochloride, amlodipine, ascorbic acid, bisoprolol, calcium folinate, cetirizine, citalopram, escitalopram, clindamycin, cyclophosphamide, diazepam, diethylcarbamazine dihydrogen citrate, digoxin, donepezil, doxazosin, doxycycline hydrochloride, fluconazole, lamivudine, levodopa, levofloxacin, levonorgestrel, lithium carbamate, loratadine, DL-methionine, mirtazapine, nicotinamide, norethisterone, ofloxacin, ondansetron, paracetamol (also called acetaminophen), phenobarbital, phenoxymethylpenicillin potassium, potassium iodide, pravastatin, promethazine hydrochloride, propylthiouracil, pyridoxine hydrochloride, quinapril, quinine sulfate, ramipril, riboflavin, salbutamol sulfate, abacavir, alendronic acid, amodiaquine, anastrazole, benznidazole, biperiden hydrochloride, captopril, carbidopa, cefaclor, chlorambucil, chloramphenicol, chlorphenamine hydrogen maleate, chlorpromazine hydrochloride, cimetidine, ciprofloxacin hydrochloride, clavulanic acid, clomiphene citrate, clomipramine hydrochloride, cloxacillin sodium, colchicine, cycloserine, didanosine, ergocalciferol, ergotamine tartrate, ethinylestradiol, ethionamide, ethosuximide, ferrous salts, flucytosine, gabapentin, glyceryl trinitrate, hydralazine hydrochloride, hydrochlorothiazide, isoniazid, isosorbide dinitrate, letrozole, levamisole hydrochloride, levothyroxine sodium, lisinpril, losartan, medroxyprogesterone acetate, metformin hydrochloride, methotrexate sodium, methyldopa, sertraline, sildenafil, tamoxifen citrate, terbinafine, theophylline, tramadol, valproic acid, venlafaxine, warfarin sodium, zolpidem, morphine sulfate, bromide bostigmine, nifurtimox, penicillamine, pentamine, procarbazine hydrochloride, pyridostigmine bromide, risedronic acid, sulfadoxine, terazosin, thiamine hydrochloride, topiramate, zinc sulfate and mixtures thereof.

Preferably, the molecule of therapeutic or cosmetic interest is selected from the group consisting of vitamins such as ascorbic acid, antimicrobial peptides such as temporins, antiviral agents and antibiotics.

### Method for Preparing Particles According to the Invention

The invention also relates to a method for preparing the particles as defined above, comprising mixing:
at least one compound of formula Chem. (I) as defined above;
at least one cyclodextrin, preferably α-cyclodextrin; and
optionally a molecule of therapeutic or cosmetic interest as defined above.

In a preferred embodiment, the compound of formula Chem. (I) and the cyclodextrin are introduced in a molar ratio compound of formula Chem. (I):cyclodextrin ranging from 1:1 to 1:20, preferably from 1:2 to 1:15, preferentially from 1:5 to 1:10.

The mixture of at least one compound of formula Chem. (I), at least one cyclodextrin, and optionally a molecule of therapeutic or cosmetic interest can be carried out in water and/or a water-miscible solvent, preferably in a polar protic solvent such as water, ethanol or a mixture thereof, preferably water.

Said mixture can be carried out at room temperature, that is to say without any heating or cooling step. It may also be considered to carry out this mixture at another temperature, for example at a temperature ranging from 0° C. to 70° C.

It can also be carried out for a period of 48 hours to 96 hours, preferably with stirring.

The method for preparing the particles according to the invention is therefore very simple since it can be carried out in a single step, by simply mixing the constituents of the particles. Furthermore, it can be carried out in a non-toxic solvent such as water.

The particles thus prepared can be used as such, that is to say suspended in the reaction medium such as water, or recovered or dried before use, for example by freeze-drying.

### Cosmetic or Pharmaceutical Composition

The present invention also relates to a cosmetic or pharmaceutical composition comprising particles as defined above and at least one cosmetically or pharmaceutically acceptable excipient. Preferably, the composition according to the invention is in a form suitable for topical application.

Galenic forms used for topical application include lotions, milks, emulsions, serums, balms, masks, creams, dispersions, gels, foams, shampoos, conditioners, and sprays.

The compositions according to the invention may contain surfactants, complexing agents, preservatives, antioxidants, stabilizing agents, emulsifiers, thickeners, gelling agents, humectants, emollients, trace elements, perfumes, colorants, UV filters and mixtures thereof.

### Use of the Particles and Compositions According to the Invention

The particles and compositions comprising at least one molecule of therapeutic or cosmetic interest can be used for the sustained release of said molecule of therapeutic or cosmetic interest.

Particles not comprising a molecule of therapeutic or cosmetic interest can also be used as such as a drug since the monosaccharide residue can act as an active ingredient. For example, particles according to the invention comprising mannose can be used to compete with the mannose naturally present on the surface of cells and which serves for the adhesion of pathogenic microorganisms such as bacteria. Therefore, such particles could be used for their antibacterial properties.

### FIGURES

FIG. 1: Chemical structure of α-CD.

FIG. 2: Macroscopic appearance of formulations A, B, E, G and I at 10 mg/mL of ML before and after 3 days of stirring at 25° C.

FIG. 3A: Images obtained in TEM and size distributions of suspensions A, B and C obtained by analysis of TEM images and adjusted by a log-normal distribution. The average is called D_{TEM}.

FIG. 3B: Images obtained in TEM and size distributions of suspensions D, E and F obtained by analysis of TEM images and adjusted by a log-normal distribution. The average is called D_{TEM}.

FIG. 3C: Images obtained in TEM and size distributions of suspensions G, H and I obtained by analysis of TEM images and adjusted by a log-normal distribution. The average is called D_{TEM}.

FIG. 4: Intensity size distribution of particles consisting of ML/α-CD (10/77 mg/mL).

FIG. 5: Effect of the initial concentration of α-CD at a concentration in ML of 10 mg/mL on Dₕ (top right diagram), D_{TEM} (top left) and Zeta potential (bottom) of particles (n=3).

FIG. 6: SEM images of particles consisting of ML/α-CD (formulation F, 10/96 mg/mL).

FIG. 7: Variation of dynamic viscosity as a function of shear rate of particle suspensions consisting of ML/α-CD (formulation E, 10/80 mg/mL and formulation I, 10/140 mg/mL).

FIG. 8: Macroscopic appearance of the 5 mg/mL formulations in ML after 3 days of magnetic stirring at 300 rpm and 25° C.

FIG. 9: Images obtained by TEM and histograms of particle size distributions obtained by analyzing several images and adjusted by a log-normal law.

FIG. 10: SEM images of particle suspensions obtained with ML/α-CD by gradually increasing the concentration of α-CD: (A) 5/40 mg/mL, (B) 5/70 mg/mL, (C)5/80 mg/mL and (D) 5/113 mg/mL.

FIG. 11: AFM images of particle suspensions obtained with ML/α-CD 5/70 mg/mL.

FIG. 12: Intensity size distribution of particles obtained in suspensions formulated from a mixture of ML/α-CD in water. The concentration of α-CD is gradually increased: (A) 5/40 mg/mL, (B) 5/70 mg/mL, (C) 5/80 mg/mL and (D) 5/113 mg/mL.

FIG. 13: Effect of the initial concentration of α-CD at a concentration in ML of 5 mg/mL on Dₕ (top right diagram), D_{TEM} (top left) and Zeta potential (bottom) of particles (n=3).

FIG. 14: Thermogravimetric analysis of α-CD and ML/α-CD.

FIG. 15: Profile of release of ascorbic acid (AA) as a function of time.

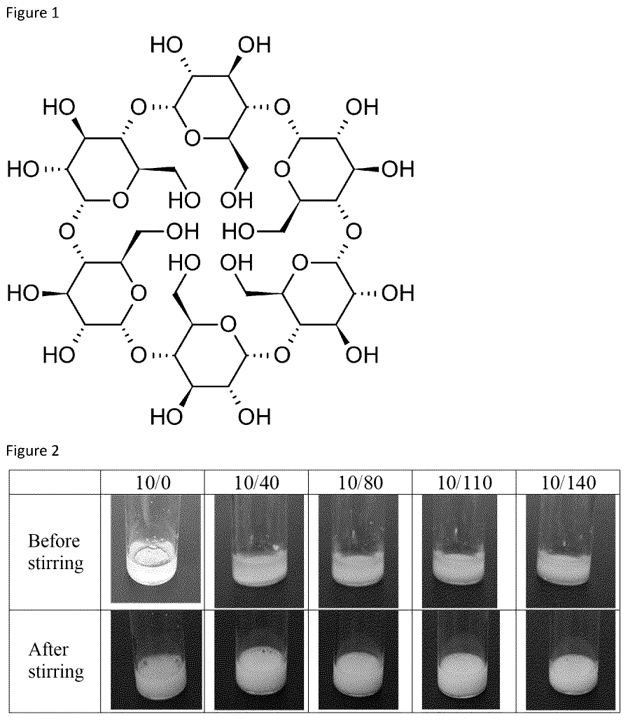

### EXAMPLES

### 1—Materials and Methods

α-CD (C₃₆H₆₀O₃₀, M=972.8 g/mol) and ascorbic acid (abbreviated AA, C₆H₈O₆, M=176.12 g/mol) were obtained from Sigma-Aldrich.

### Characterization of Mannolipid (ML)

The solubility study of ML was carried out in water, methanol and methanol/chloroform mixture 1/1 v/v. In 10 mL glass vials, 10 mg of ML were added to 500 μL of solvent and left under magnetic stirring at 300 rpm at 25° C.

Fourier Transform Infrared spectroscopy analyses were performed using a JASCO FT/IR-4100 spectrometer with a 4 cm⁻¹ resolution. An accumulation of 60 scans per measurement was performed to obtain a spectrum between 4000 and 400 cm⁻¹ analyzed by the Spectra Manager software. The sample to be analyzed is placed in contact with a crystal (diamond) before being traversed by the infrared beam.

Nuclear magnetic resonance (NMR) spectra were recorded with a 300 MHz Bruker UltraShield spectrometer.

### Physicochemical Characterizations of Particles

Particle size measurement was investigated by dynamic light scattering (DLS) using a Zetasizer Nanoseries (Malvern, France). For this purpose, 40 μL of each particle suspension is diluted in 960 μL of MillQ® water before detection. Each sample is analyzed three times at 25° C. Detection is performed at 173°.

The Zeta potential of the particles was obtained on the same apparatus by diluting the particle suspensions as described previously; 40 μL were dispersed in 960 μL of a 1 mM NaCl solution.

Each suspension was then placed in a Zeta potential measuring cell. For each sample, the Zeta potential corresponds to the average of three successive measurements.

Transmission electron microscopy (TEM) was used to determine the morphology and size of the particles obtained. The microscope used was a JEOL 1400 operating at 80 kV (Imagif, Gif sur Yvette, France). Each suspension (1 μL) was diluted in 49 μL of MilliQ® water, then 4 μL of this diluted suspension was deposited on a carbon-coated copper grid. After 5 min of drying, the grid was introduced into the microscope to visualize the sample. The size distribution was determined by analyzing at least 5 images of each sample using Image J software.

Scanning electron microscopy (SEM) images were obtained using a PEG electron microscope (Merlin model). Samples were diluted 1/100 (1 μL of the particle suspension in 99 μL of MillQ® water), then 25 μL of this dilution was placed on a metal pad and left to dry for 24 h before observation.

Atomic force microscopy (AFM) observations were performed using an Asylum MFP-3D microscope (Oxford Instruments, USA) allowing observations in air and liquid media. AFM images were recorded in a non-contact mode with a silicon tip coated with an aluminum layer oscillating at a control frequency of 270 kHz. The suspension (1 μL) was diluted in 39 μL of MilliQ® water, then 2 μL of this diluted suspension was deposited on a mica plate and left to dry for 10 min.

### Rheological Characterizations of Particle Suspensions

Rheological tests were conducted with a rotary absolute rheometer AR-G2 (TA Instruments) equipped with a 40 mm diameter parallel-plate cell and a Peltier device that allows temperature control (T=20° C.). The air gap angle was set at 1° and the surfaces were roughened to reduce the risk of wall slippage. Flow curves were obtained by applying a shear rate between 1 and 2000 s⁻¹.

Quantity of α-CD Interacting with the Alkyl Chains of the Mannolipid (ML)

Formulations J to M (see Table 2) were centrifuged (Thermo Scientific Sorvall ST 16R Centrifuge) for 30 min at 3000 G to separate the assemblies from the supernatant. The supernatant, containing excess α-CD, was recovered and filtered (0.2 μm hydrophilic syringe filter) to remove residual assemblies and aggregates larger than 0.2 μm. The filtrate obtained was subjected to a polarimetric assay (λ=589 nm, Perkin Elmer 341 polarimeter) to quantify the amount of α-CD that had not interacted with ML.

### Encapsulation of Ascorbic Acid

In order to estimate the encapsulation capacity of the particles according to the invention, vitamin C, also called ascorbic acid (AA), was tested as a hydrophilic model molecule. For this purpose, three 2 mL solutions of AA at different concentrations (1 mg/mL, 2 mg/mL and 3 mg/mL) were prepared. Then, 5 mg of ML and 70 mg of α-CD were introduced in a small vial protected from light, then the mass is adjusted to 1 g with the AA solution. The suspension is left stirring for 3 days at 25° C. at 300 rpm. To determine the encapsulation efficiency (EE), the formulations were centrifuged at 3000 G for 30 min and the concentration of free (non-encapsulated) AA was determined at a wavelength equal to 265 nm using a spectrophotometer (Perkin-Elmer Lambda 25 controlled by UVWinlab software).

### Release of Ascorbic Acid

The release of AA from the particles was studied in phosphate buffer saline (PBS, pH=7.4±0.45) using the dialysis method. After removing the supernatant to determine the EE, the AA-loaded particles (ML/(α-CD 5/70 mg/mL formulation with an initial AA concentration of 3 g/L) were freeze-dried then redispersed (m=17 mg) in 2 mL of phosphate buffer and introduced into a dialysis bag (MWCO=3500 Da) which was subsequently immersed in a beaker containing 400 mL of PBS. The beaker was kept at 25° C. and stirred with a magnetic stir bar. At regular time intervals, 2 mL of the PBS was withdrawn for UV analysis (λ=265 nm) to determine the concentration of released AA, and the same volume of fresh PBS was added to the release medium.

For freeze-drying, 500 μL of the particle suspension was placed in a vial then frozen at −20° C. for 12 h. The sample was then placed in the freeze-dryer chamber for 24 h at −55° C. under a pressure of 0.5 mbar.

### 2—Synthesis of Mannolipids

### 2.1. Synthesis of ML

### Synthesis and characterization of 3-azidopropyl-2,3,4,6-tetra-O-acetyl-α-D-mannopyranoside

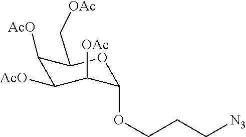 [SMILES: CC(=O)OCC1OC(OCCCN=[N+]=[N-])C(OC(C)=O)C(OC(C)=O)C1OC(C)=O]

This compound is synthesized from mannose pentaacetate (CAS RN 4163-65-9) and 3-chloropropanol in two steps as described in the literature (Ma et al. 2016).

¹H NMR (300 MHz, CDCl₃): δ 5.34-5.13 (m, 4H), 4.75 (d, J=1.7 Hz, 1H), 4.21 (dd, J=12.2, 5.4 Hz, 1H), 4.05 (dd, J=12.2, 2.5 Hz, 1H), 3.90 (ddd, J=8.3, 5.4, 2.4 Hz, 1H), 3.80-3.70 (m, 1H), 3.46 (dt, J=9.9, 5.9 Hz, 1H), 3.37 (t, J=6.5 Hz, 2H), 2.09 (s, 3H, CH₃Ac), 2.04 (s, 3H, CH₃Ac), 1.98 (s, 3H, CH₃Ac), 1.93 (s, 3H, CH₃Ac), 1.89-1.76 (m, 2H).

¹³C NMR (75 MHz, CDCl₃): δ 170.7 (CO Ac), 170.2 (CO Ac), 170.0 (CO Ac), 169.8 (CO Ac), 97.8, 69.6 (C_{Man}), 69.2 (C_{Man}), 68.8 (C_{Man}), 66.3 (C_{Man}), 65.0, 62.6, 48.2, 28.8, 21.0 (CH₃Ac), 20.8 (CH₃Ac), 20.8 (CH₃Ac), 20.8 (CH₃Ac).

### Synthesis and Characterization of Tetraacetylated Mannolipid

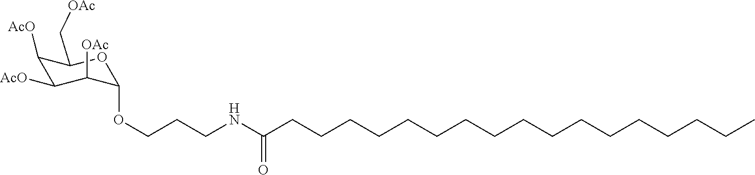 [SMILES: CCCCCCCCCCCCCCCCCC(=O)NCCCOC1OC(COC(C)=O)C(OC(C)=O)C(OC(C)=O)C1OC(C)=O]

Triphenylphosphine (543 mg, 2.074 mmol, 1.3 equiv.) was added to a solution of 3-azidopropyl-2,3,4,6-tetra-O-acetyl-α-D-mannopyranoside (688 mg, 1.595 mmol, 1 equiv.) and stearoyl chloride (773 mg, 2.552 mmol, 1.6 equiv.) in anhydrous dichloromethane (DCM) (2 mL). The mixture was stirred overnight and N,N-diisopropylethylamine (330 mL, 1.914 mmol, 1.2 equiv.) was added. Stirring was stopped 15 minutes after the addition of N,N-diisopropylethylamine (DIEA) by diluting with dichloromethane (13 mL) and washing with 1M hydrochloric acid solution (2×5 mL) and saturated sodium bicarbonate solution (5 mL). The organic phase was dried over magnesium sulfate, filtered, and concentrated under reduced pressure. The residue was then roughly purified by flash chromatography (CyHex/AcOEt 60:40) to obtain 1 gram of tetraacetylated mannolipid as the major compound.

Rf: 0.23 (CyHex/AcOEt 60:40).

¹H NMR (300 MHz, CDCl₃): δ 5.97 (t, J=5.1 Hz, 1H, NH amide), 5.24-5.19 (m, 2H), 5.16 (app s, 1H), 4.74 (s, 1H), 4.22 (dd, J=12.2, 5.1 Hz, 1H), 4.02 (dd, J=12.2, 2.0 Hz, 1H), 3.91 (br s, 1H), 3.78-3.64 (m, 1H), 3.44 (dt, J=10.0, 5.9 Hz, 1H), 3.36-3.22 (m, 2H), 2.15-2.06 (m, 5H, NHCOCH₂, CH₃Ac), 2.03 (s, 3H, CH₃Ac), 1.98 (s, 3H, CH₃Ac), 1.92 (s, 3H, CH₃Ac), 1.83-1.71 (m, 2H), 1.60-1.49 (m, 2H, NHCOCH₂CH₂), 1.18 (br s, 28H, 14×CH₂), 0.80 (t, J=6.4 Hz, 3H).

¹³C NMR (75 MHz, CDCl₃): δ 173.4 (CO amide), 170.6 (CO Ac), 170.0 (CO Ac), 169.9 (CO Ac), 169.6 (CO Ac), 97.6, 69.4, 69.1, 68.5, 66.5, 66.1, 62.5, 37.0, 36.7 (NHCOCH₂), 31.9, 29.7-29.3 (13×CH₂), 29.3, 25.8 (NHCOCH₂CH₂), 22.7 (CH₂), 20.8 (CH₃Ac), 20.7 (CH₃Ac), 20.7 (CH₃Ac), 20.6 (CH₃Ac), 14.1 (CH₃).

### Synthesis and Characterization of Mannolipid ML

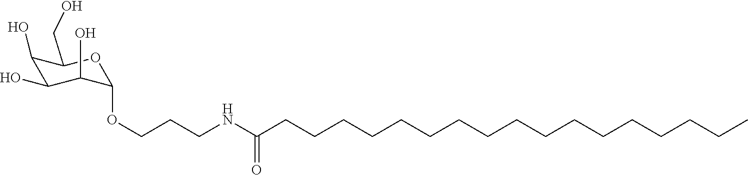 [SMILES: CCCCCCCCCCCCCCCCCC(=O)NCCCOC1OC(CO)C(O)C(O)C1O]

Sodium (30 mg) was added to a solution of tetraacetylated mannolipid in anhydrous methanol (30 mL) at 0° C. The solution was mixed for 5 hours while allowing the temperature to rise to room temperature then the mixture was neutralized using a cation exchange resin such as Amberlyst® IR 120 H+. The resin is then removed by filtration and the filtrate is then concentrated under reduced pressure. The crude product is then purified by column chromatography on silica gel (DCM/EtOH 85:15) to obtain the pure product (390 mg, 49%, yields of both steps) in the form of a white powder.

Rf: 0.26 (DCM/EtOH 85:15).

ESI-HRMS m/z [M+H]+ calculated for C₂₇H₅₄NO₇: 504.3895, found: 504.3890.

¹H NMR (300 MHz, MeOD/CDCl₃ 90:10 v:v): δ 4.75 (s, 1H), 3.93-3.21 (m, 10H), 2.22 (t, J=7.3 Hz, 2H), 1.87-1.76 (m, 2H), 1.62 (br s, 2H), 1.29 (br s, 28H, 14×CH₂), 0.98-0.84 (m, 3H, CH₃).

¹³C NMR (75 MHz, MeOD/CDCl₃ 90:10 v:v): δ 175.3 (CO amide), 100.2, 73.2 (C_{Man}), 71.3 (C_{Man}), 70.7 (C_{Man}), 67.2 (C_{Man}), 64.6, 61.6, 36.5, 35.7, 31.7, 29.5-28.9 (13×CH₂), 25.8, 22.4, 13.3.

### 2.2. Alternative Synthesis of ML

### 3-chloropropyl-2,3,4,6-tetra-O-benzoyl-α-D mannopyranoside

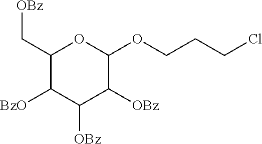

BF₃·Et₂O (0.131 mL) is added to a solution of mannose perbenzoate (1.0 g) in anhydrous dichloromethane (12 mL). The solution is stirred for 15 h, neutralized with an aqueous solution of sodium bicarbonate. The organic phase is separated and dried (MgSO₄). The crude product is then purified by chromatography on a silica gel column (80/20 cyclohexane/EtOAc) to obtain 0.676 g of pure product (67% yield).

### 3-azidopropyl-2,3,4,6-tetra-O-benzoyl-α-D mannopyranoside

A solution of the compound obtained in the previous step (0.7 g) in anhydrous DMF (10 mL) is heated at 70° C. for 7 h in the presence of sodium azide (0.405 g). The reaction medium is concentrated, the residue is taken up in water and dichloromethane. The organic phase is dried (MgSO₄) and concentrated under reduced pressure.

### 3-octadecanoylamino-2,3,4,6-tetra-O-benzoyl-α-D mannopyranoside

The previous compound (0.40 g) is dissolved in a THE (2 mL)/Water (12 μL) mixture in the presence of triphenylphosphine (0.2 g) and stirred at 45° C. for 15 h. The mixture is concentrated under reduced pressure and the residue is dissolved in dichloromethane (1 mL). The solution is cooled to 0° C., di-isopropylamine DIPA (0.123 mL) is added, followed by stearoyl chloride (0.28 g). The solution is then stirred at room temperature for 16 h, then concentrated to dryness. The crude product is then purified by silica gel column chromatography (80/20 cyclohexane/EtOAc) to obtain 0.2 g of pure product.

### 3-octadecanoylamino-α-D mannopyranoside

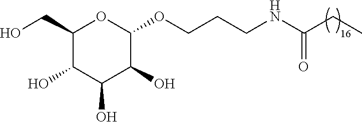

The product obtained in the previous step (0.2 g) is dissolved in anhydrous methanol (3 mL) in the presence of a catalytic amount of sodium. The solution is stirred at room temperature for 15 h, then it was neutralized using a cation exchange resin such as Amberlyst® IR 120 H+. The solution is then concentrated to dryness. The residue is then purified by chromatography on a silica gel column (60/40 cyclohexane/EtOAc then 80/20 EtOAc/MeOH) to obtain 70 mg of pure product (65%).

### 2.3. Synthesis of 3-hexadecanoylamino-α-D mannopyranoside

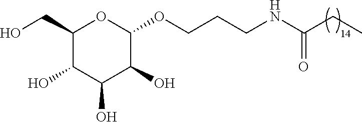

This compound is prepared using the method described in Example 2.2. from 3-azidopropyl-2,3,4,6-tetra-O-benzoyl-α-D mannopyranoside and substituting the stearoyl chloride with palmitoyl chloride.

¹H NMR (300 MHz, MeOD/CDCl₃ 90:10 v:v): δ 4.74 (s, 1H), 3.93-3.21 (m, 10H), 2.22 (t, J=7.3 Hz, 2H), 1.87-1.76 (m, 2H), 1.62 (br s, 2H), 1.29 (br s, 24H, 12×CH₂), 0.98-0.84 (m, 3H, CH₃).

### 3—Preparation of the Particles According to the Invention

Mannolipid, the synthesis of which is described above (ML), alpha-cyclodextrin (α-CD) and MilliQ® water were successively introduced into 10 mL glass vials. The suspensions were left under magnetic stirring at 300 rpm for 72 h at 25° C.

Several particle syntheses were performed at different concentrations of α-CD and ML (see Table 2).

TABLE 2

| | | | MilliQ ® | | |
|---|---|---|---|---|---|
| Formu- | ML | α-CD | Water q.s. | ML/α-CD | Rₘₒₗₑₛ |
| lation | (mg) | (mg) | 1 g (g) | (mg/mL) | α-CD/ML |
| A | 10 | 0 | 0.989 | 10/0 | 0.0 |
| B | 10 | 40 | 0.950 | 10/40 | 2.1 |
| C | 10 | 57 | 0.931 | 10/57 | 3.0 |
| D | 10 | 77 | 0.911 | 10/77 | 4.0 |
| E | 10 | 80 | 0.910 | 10/80 | 4.1 |
| F | 10 | 96 | 0.892 | 10/96 | 5.0 |
| G | 10 | 110 | 0.880 | 10/110 | 5.7 |
| H | 10 | 134 | 0.854 | 10/134 | 6.9 |
| I | 10 | 140 | 0.850 | 10/140 | 7.2 |
| I | 5 | 40 | 0.955 | 5/40 | 4.1 |
| K | 5 | 70 | 0.925 | 5/70 | 7.2 |
| L | 5 | 80 | 0.915 | 5/80 | 8.3 |
| M | 5 | 113 | 0.882 | 5/113 | 11.7 |

Rα-CD/ML is the ratio of the number of moles of α-CD to ML.

### Compositions of Prepared Particle Suspensions

### 4—Characterization of α-CD/ML Particles

### 4.1. 10 mg/mL Formulations (A to I)

### Macroscopic Aspect:

Without α-CD, ML is insoluble in water before stirring. After 72 h of stirring, a white suspension is obtained. After a few hours without stirring, a precipitate forms, which may be the sedimentation of water-insoluble ML aggregates. By adding α-CD, whitish and opaque suspensions were obtained by the interaction of the alkyl chains (C₁₇) of ML with α-CD (see FIG. 2).

### Morphology, Size and Load:

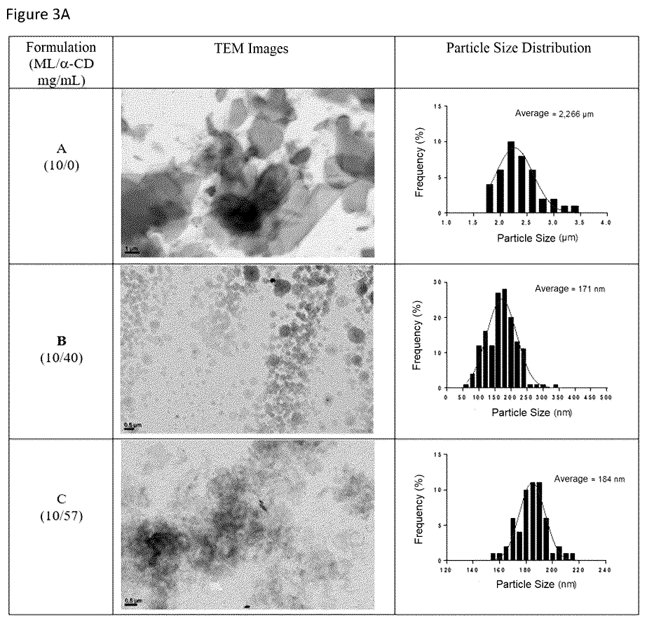

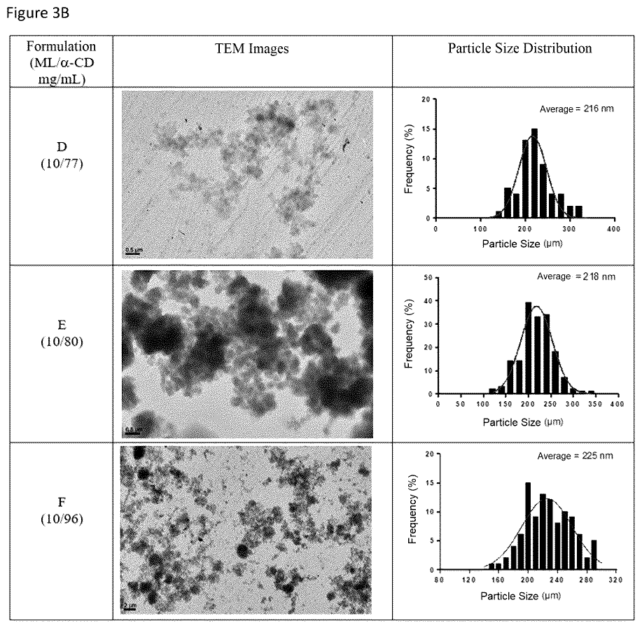

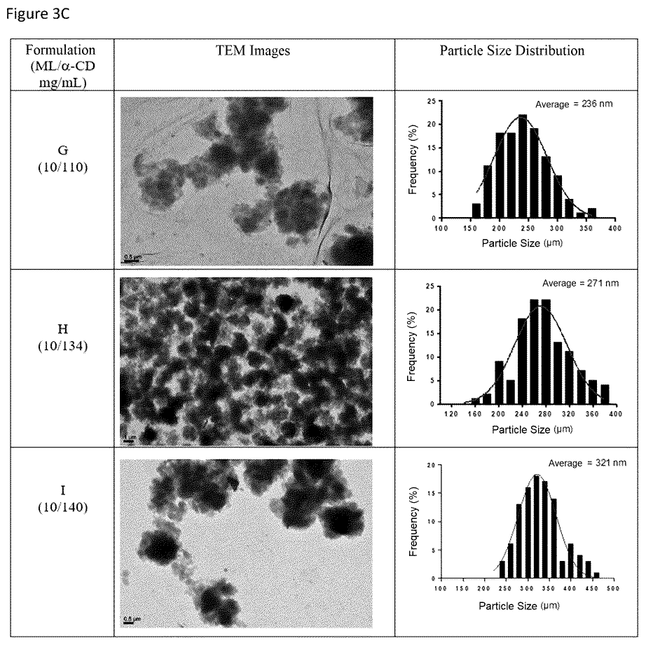

The formulations were first observed by TEM. Without α-CD (10/0), the image in FIG. 3A shows objects of indefinite shape and aggregates larger than 2 μm, which are highly polydisperse. The size of these aggregates can reach 3.4 μm. This may be due to the low solubility of ML in water. In the presence of α-CD (formulation B, 40 mg/mL), the TEM image shows objects ranging in size from 50 to 350 nm. The morphology of the particles is a rather rounded shape.

As the concentration of α-CD increases, the particles become more contrasted and the particle shape becomes more visible. In the presence of α-CD at concentrations above 80 mg/mL, TEM images of the particles formed in the different formulations highlight small spherical particles grouped around larger particles. FIGS. 3B and 3C show that increasing the initial amount of α-CD and therefore the ratio R_{α-CD/C17} leads to an increase in the average particle diameter analyzed by TEM (D_{TEM}). The higher the concentration of α-CD, the greater the number of α-CD molecules interacting with the ML. As a result, the alkyl chains of the ML become saturated with α-CDs leading to larger, organized and well-defined structures.

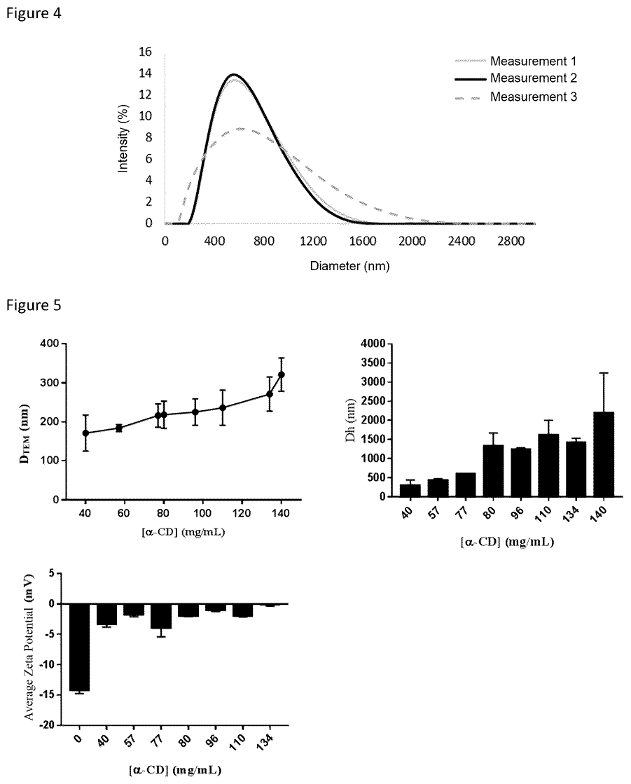

The hydrodynamic diameter of the particles suspended in water was subsequently measured by dynamic light scattering (DLS). FIG. 4 highlights the intensity distribution of particle diameters in formulation D (10/77 mg/mL). This figure shows that the particles have a diameter of approximately 608±7 nm with a polydispersity index (PDI) of 0.245.

The results of the measurements of the variation of hydrodynamic diameters as a function of the α-CD concentration are shown in FIG. 5. The particle sizes measured by TEM are much smaller than those obtained by DLS. This discrepancy between the DLS and TEM values becomes even more marked for α-CD concentrations higher than 57 mg/mL for which the formulations are more opaque and viscous.

In addition, it can be seen that the average particle diameter in the formulation 10/77 mg/mL (D_{TEM}=216±30 nm) is much lower than that measured by DLS (Dₕ=608±7 nm). As can be seen in TEM images, the particles are adhered to each other and assembled into aggregates. As a result, their scattering intensity is stronger than that of individual particles and the value of the hydrodynamic diameter obtained is only a measure of the diameter of several aggregated particles.

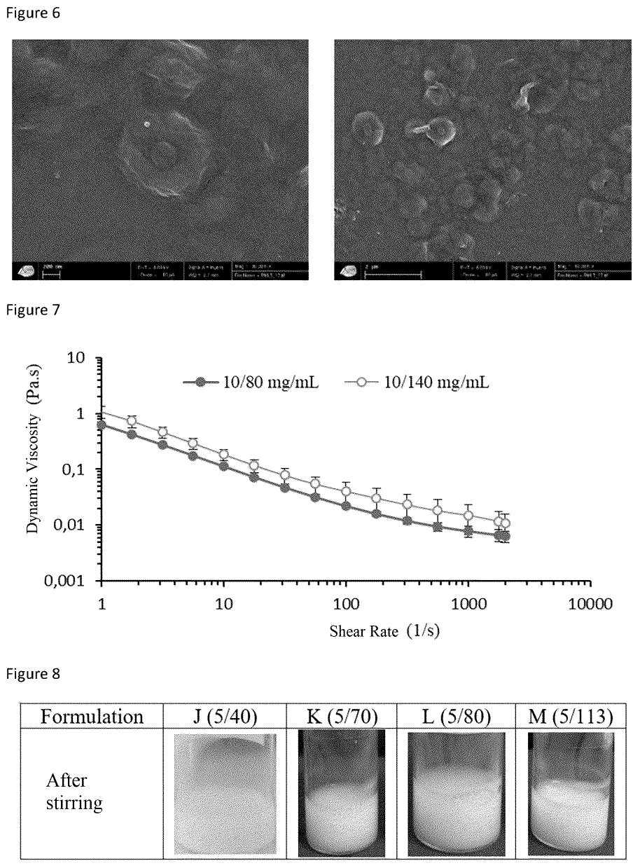

Regarding the Zeta potential of the particles, it can be seen that they have a negative Zeta potential. An increase in the Zeta potential with the concentrations of α-CD can also be seen. The latter starts at −14.22±0.22 mV in the absence of α-CD to then reach a value of −0.12±0.25 mV in formulation I 10/140 mg/mL. This result is attributed to the threading of the ML alkyl chains into the CD cavities, which leads to a masking of their negative charge and therefore an increase in the charge of the particles. It should be noted that although the general trend is towards an increase in the Zeta potential with increasing α-CD concentrations, the potentials obtained remain close in suspensions whose α-CD concentration varies from 57 to 134 mg/mL with an average value of −2.83±1.10 mV and an average deviation of −0.89 mV. The SEM images of formulation F shown in FIG. 6 highlight spherical particles. It was observed that some particles had a structure with a core and a crown. This could be the result of two successive processes. First, the α-CDs will thread along the alkyl chain of the ML to form inclusion complexes which then self-assemble into a bilayer. Indeed, the ML molecules have a polar head (mannose) and an apolar hydrocarbon chain. The polar parts would remain in contact with the external aqueous medium while the interior of the bilayer is composed of alkyl chains included in the hydrophobic cavities of the α-CD molecules.

### Rheological Properties:

Rheological properties were determined by measuring dynamic viscosity as a function of shear rate of 10/80 mg/mL and 10/140 mg/mL formulations (FIG. 7). It is noted that the viscosity decreases with the shear rate. The particle suspensions therefore have a shear-thinning behavior. This phenomenon could result from the disruption of interactions between the particles on the one hand and the interruption of the non-covalent bond points within the inclusion complexes under the effect of the applied stress on the other hand. In addition, it is noted that the viscosity value increases with the increase in the concentration of α-CD. This behavior can be explained by the increase in particle size (increase in the volume fraction occupied by the particles), which opposes the flow.

### 4.2. 5 mg/mL ML Formulations (J to M)

### Macroscopic Aspect:

After 3 days of stirring, the suspensions obtained are white (FIG. 8) and much less viscous than those obtained with a ML concentration of 10 mg/mL.

### Morphology, Size and Load:

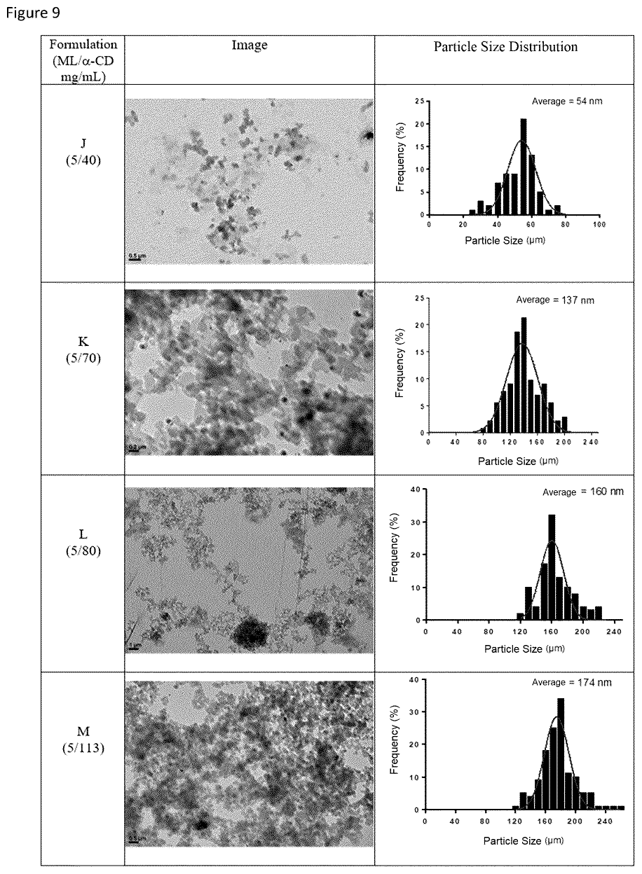

In order to characterize the morphology and size of the particles formed, a TEM study of these formulations was conducted. The observed images are dominated by the presence of spherical particles aggregated with each other (FIG. 9). In addition, it is observed that the increase in the concentration of α-CD is accompanied by an increase in the particle size. This result is in agreement with what was observed with the particles obtained at a ML concentration of 10 mg/mL.

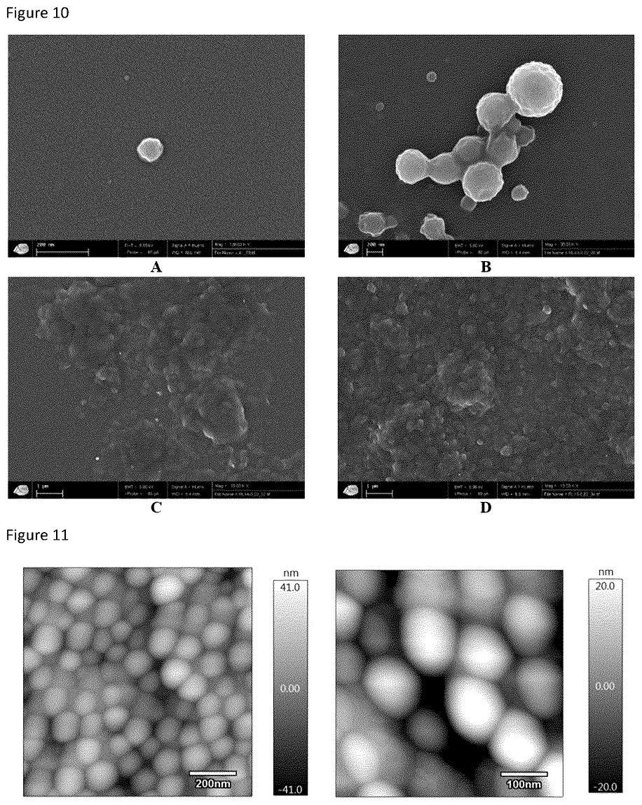

The particles were also characterized by SEM and AFM. As shown in FIG. 10 obtained by SEM, the particles obtained are spherical. The AFM images of the particle suspension obtained with ML/(α-CD 5/70 mg/mL in FIG. 11 show the presence of spherical nanoparticles with a diameter of approximately 130 nm close to that measured by TEM.

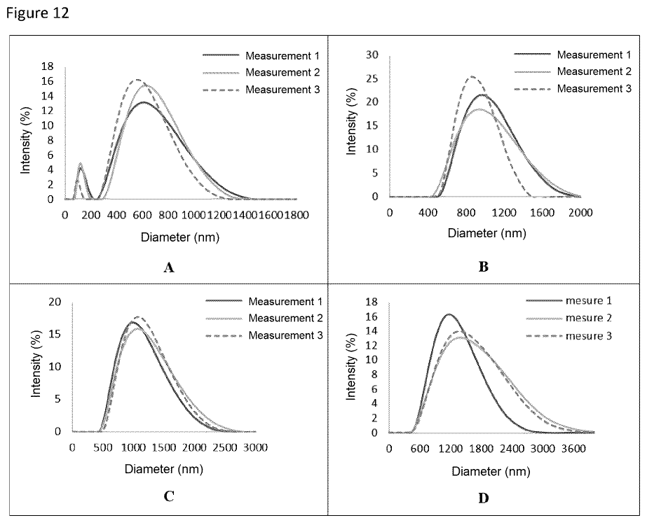

DLS measurements were used to determine the hydrodynamic diameters of particles in solution. FIG. 12. A shows particles of approximately 100 nm accompanied by particles of size equal to 628 nm. The first peak could correspond to well-dispersed individual particles while the second would be attributed to aggregated particles. In contrast, the intensity distributions of particle diameters obtained at 70, 80 and 113 mg/mL of α-CD, have a single peak at 920±32 nm, 1114±45 nm and 1174±125 nm respectively.

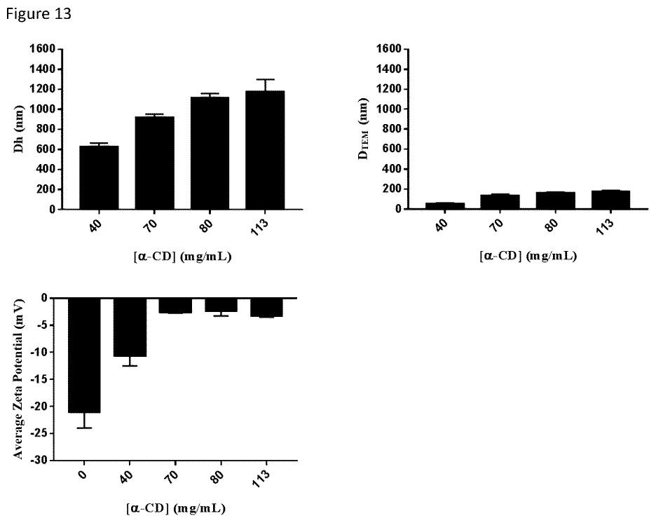

As for the particles obtained with an ML concentration of 10 mg/mL, the values of the hydrodynamic diameters are clearly higher than those obtained by MET, SEM and AFM microscopy (FIG. 13).

The zeta potential measurements of the particles presented in FIG. 13 show that it is negative in the absence of α-CD. The zeta potential increases significantly from a concentration of 40 mg/mL of α-CD (−10.60±1.91 mV) to 70 mg/mL (−2.54±0.2 mV). From an α-CD concentration of 70 mg/mL, the zeta potential hardly varies.

A polarimetric assay of the supernatants obtained after centrifugation and filtration was carried out. Table 3 lists the results of the initial concentrations of α-CD ([α-CD]₀) as well as the residual concentrations of α-CD ([α-CD]_{R}). The values of [α-CD]_{R} were obtained from the measurements of the rotation angles made on the supernatants. The concentration of α-CD interacting with the alkyl chains of the ML ([α-CD]_{interaction}) is then deduced by subtraction between the initial concentrations and those found in the filtrate.

TABLE 3

| | [α- | Angle | [α- | | |
|---|---|---|---|---|---|
| [ML] | CD]₀ | of | CD]_{R} | [α- | Ratio |
| (mg/ | (mg/ | rotation | (mg/ | CD]_{interaction} | (n(_{alkyl chain)})/ |
| mL) | mL) | (°) | mL) | (mg/mL) | (n_{(α-CD)interaction}) |
| 5 | 40 | 0.57 | 12.30 | 27.69 | 1/1.42 |
| 5 | 70 | 1.82 | 39.33 | 30.67 | 1/1.57 |
| 5 | 80 | 2.26 | 48.84 | 31.16 | 1/1.6 |
| 5 | 113 | 3.74 | 80.83 | 34.16 | 1/1.75 |

n_{(alkyl chain)}: number of moles of C17 chains that would be likely to interact with the α-CD; n_{(α-CD)interaction}: number of moles of α-CD that would interact with the alkyl chains (C17) carried by the ML

### Summary of the Results of α-CD Assays in Suspension Supernatants

Increasing the initial concentration of α-CD leads to an increase in the stoichiometry of the interaction between the C17 alkyl chain and the α-CD. The higher the concentration of α-CD, the more molecules interact with the alkyl chain of the ML and saturate more with α-CD, which may explain the increase in particle size observed during TEM analysis.

### 4.4. Thermogravimetric Study

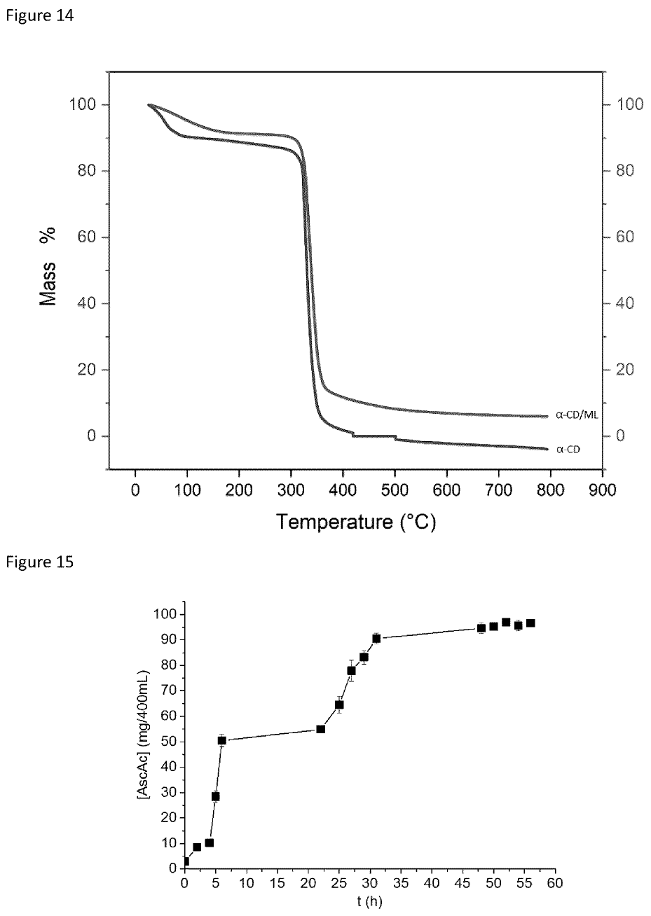

The thermal behavior of ML/α-CD at 5 mg·mL⁻¹ and 70 mg·mL⁻¹ respectively and uncomplexed α-CD were studied using thermogravimetric analysis (FIG. 14). The initial weight loss observed in α-CD below 120° C. is due to the removal of bound water molecules in the α-CD cavity. In contrast to the decomposition observed in uncomplexed α-CD, the ML embedded in α-CD showed a gradual decomposition from 100° C.-250° C., along with a shift and a wider decomposition temperature range. This can be attributed to the presence of the guest molecule ML instead of the water molecule in the cavity. The dip observed between 200° C. and 360° C. is due to the decomposition of CD. Interestingly, in the case of uncomplexed α-CD, the residual mass percentage reaches 0 at 420° C. A residual mass of 10.80% is observed in the case of α-CD/ML. This change in decomposition behavior can be attributed to the stable inclusion of the host and possible intermolecular bonding between CDs.

### 5—Encapsulation and Release of Ascorbic Acid (AA)

The encapsulation of AA within particles is based mainly on its non-covalent interaction (hydrophobic interactions, hydrogen bonds, Van der Waals forces) with the particles. Table 4 lists the encapsulation efficiencies of particles formed with ML/α-CD (5/70 mg/mL).

TABLE 4

| [AA]ᵢₙᵢₜᵢₐₗ | [AA]encapsulated | EE |
|---|---|---|
| (mg/mL) | (mg/mL) | (%) |
| 1 | 0.39 | 39 |
| 2 | 0.57 | 29 |
| 3 | 0.96 | 32 |

$$E E : Encapsulation efficiency ( \% ) = {[ AA ]}_{encapsulated} {/ [ AA ]}_{initial} \times 100$$

### Particle Encapsulation Efficiency as a Function of Initial AA Concentration

The concentration of encapsulated AA increases from 0.39 to 0.96 mg/mL when the initial concentration varies from 1 to 3 mg/mL. These results show that it is possible to encapsulate a hydrophilic molecule in the particles.

The release of AA from particles containing 3 mg/mL of AA was then studied in a phosphate buffer saline (PBS—pH 7.4±0.45) medium, thus mimicking the blood environment. It is useful to remember that the particles are more particularly intended for topical application (cutaneous and/or mucous membranes) and that this protocol is widely used in the literature for the study of the release of active substances intended for topical routes (Jing Li et al. 2011). The release profile (FIG. 15) shows several stages. A first region characterized by a slow initial release lasting 5 hours (less than 30%) and a second region characterized by a rapid release. The latter is probably related to a rapid diffusion of AA molecules adsorbed on the surface of the particles by weak bonds while the slow release at the beginning is probably due to a diffusion of AA trapped in the core of the particles. In view of these results, it can be concluded that the particles allowed to obtain, on the one hand, a controlled release of AA since the amount released at the beginning remains low and, on the other hand, a faster release after 5 hours, resulting in an increase in the release rate where a cumulative concentration of 81% is reached after 30 hours.

### BIBLIOGRAPHIC REFERENCES

Ahmed Z. et al. New insights on the structure of hexagonally faceted platelets from hydrophobically modified chitosan and α-cyclodextrin. International Journal of Pharmaceutics. 2018, 548(1), 23-33.
Crini G., Fenyvesi É., Szente L. Outstanding contribution of Professor József Szejtli to cyclodextrin applications in foods, cosmetics, drugs, chromatography and biotechnology: a review. Environmental Chemistry Letters. 2021, 19(3), 2619-2641.
Del Valle, E. M. M. Cyclodextrins and their uses: a review. Process biochemistry. 2004, 39(9), 1033-1046.
Diaz-Salmeron R., et al. Hierarchical supramolecular platelets from hydrophobically-modified polysaccharides and α-cyclodextrin: Effect of hydrophobization and α-cyclodextrin concentration on platelet formation. International Journal of Pharmaceutics. 2018, 548(1), 227-236.
Greene's Protective Groups In Organic Synthesis”, 4th edition, 2007, John Wiley & Sons, Hoboken, New Jersey
Jing Li J., Zhao F., Li J. Polyrotaxanes for applications in life science and biotechnology. Applied Microbiology and Biotechnology. 2011, 90, 427-443.
Ma J. et al. Glycosylated platinum(IV) prodrugs demonstrated significant therapeutic efficacy in cancer cells and minimized side-effects. Dalton Transactions, 2016, 45, 11830-11838.
Mathapa B. G., Paunov V. N. Self-assembly of cyclodextrin-oil inclusion complexes at the oil-water interface: a route to surfactant-free emulsions. Journal of Materials Chemistry A. 2013, 1, 10836-10846.
Mirchandani Y., Patravale V. B., Brijesh S. Solid lipid nanoparticles for hydrophilic drugs. Journal of Controlled Release. 2021, 335, 457-464.
Rekharsky M. V., Inoue Y. Complexation thermodynamics of cyclodextrins. Chemical reviews. 1998, 98(5), 1875-1918.
Salatin et al. An Alternative Approach for Improved Entrapment Efficiency of Hydrophilic Drug Substance in PLGA Nanoparticles by Interfacial Polymer Deposition Following Solvent Displacement. Jundishapur Journal of Natural Pharmaceutical Products. 2018, 13(4):e 12873. WO 2013/150193
Zhang L., Su T., He B., Gu Z. Self-assembly Polyrotaxanes Nanoparticles as Carriers for Anticancer Drug Methotrexate Delivery. Nano-Micro Letters. 2014, 6, 108-115.

1. Particles comprising:
at least one compound having the following formula: 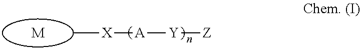
in which
n represents 0 or 1;
M represents a residue of a monosaccharide of which an OH is replaced by the group —X-(A-Y)_{n-Z} and of which one or more —OH groups are optionally replaced by a group —OSO₃H and/or of which a group —CH₂—OH is optionally replaced by a group —COOH;
X represents —O—, —S—, —NR₁—, —OC(O)—, —SC(O)—, —N(R₁)C(O)—, —OC(S)—, —SC(S)—, or —N(R₁)C(S)—, the first atom of the groups being linked to M;
A represents a linker, the linker being a (C₁-C₂₀)alkyl chain, optionally interrupted one or more times by one or more moieties selected from the group consisting of —O—, —S—, —NR₂—, —C(O)—, and —C(S)—, and/or optionally substituted with one or more groups selected from the group consisting of a halogen atom, —NO₂, —CN, —OR₃, —SR₄, and —NR₅R₆;
Y represents —O—, —S—, —NR₇—, —C(O)—, —C(S)—, —OC(O)—, —C(O)O—, —SC(O)—, —C(O)S—, —N(R₇)C(O)—, —C(O)N(R₇)—, —OC(S)—, —C(S)O—, —SC(S)—, —C(S)S—, —N(R₇)C(S)—, or —C(S)N(R₇)—;
R₁ to R₇ represent, independently of each other, a hydrogen atom or a (C₁-C₆)alkyl group; and
Z represents a saturated or unsaturated hydrocarbon chain comprising from 4 to 40 carbon atoms;
at least one cyclodextrin.

2. The particles according to claim 1, wherein M represents a residue of D-mannose, D-galactose, D-xylose or D-glucose of which one or more —OH groups are optionally replaced by a group —OSO₃H or of which a group —CH₂—OH is optionally replaced by a group —COOH.

3. The particles according to claim 1, wherein X represents —O—, —NR₁—, —OC(O)—, or —N(R₁)C(O).

4. The particles according to claim 1, wherein:
n represents 0 and X represents —N(R₁)C(O)—, N(R₁) being linked to M; or
n is equal to 1, A represents a —(CH₂)ₘ— chain with m representing 1, 2, 3, 4, 5, 6, 7 or 8, and Y represents —OC(O)—, —C(O)O—, —N(R₇)C(O)— or —C(O)N(R₇)—, the first atom of the groups being linked to A; or
n is equal to 1, X represents —O—, A represents a —(CH₂)ₘ— chain with m representing 1, 2, 3, 4, 5, 6, 7 or 8, and Y is —N(R₇)C(O)— or —C(O)N(R₇)—; or
n is equal to 1, X represents —O—, A represents —CH₂CH₂—(O—CH₂CH₂)ₚ—, p is 1, 2, 3, 4 or 5, in particular 1, and Y represents —N(R₇)C(O)—, with N(R₇) being linked to A.

5. The particles according to claim 1, wherein:
M represents a residue of mannose, galactose, xylose or glucose, one or more —OH groups of which are optionally replaced by a group —OSO₃H and/or one group —CH₂—OH of which is optionally replaced by a group —COOH;
n is equal to 1;
A represents a —(CH₂)ₘ— chain with m representing 1, 2, 3, 4, 5 and 6, and Y represents —OC(O)—, —C(O)O—, —N(R₇)C(O)— or —C(O)N(R₇)— with R₇ as defined in claim 1; and
Z represents a saturated or unsaturated hydrocarbon chain comprising from 12 to 24 carbon atoms.

6. The particles according to claim 1, wherein the particles comprise:
at least one compound selected from the group consisting of 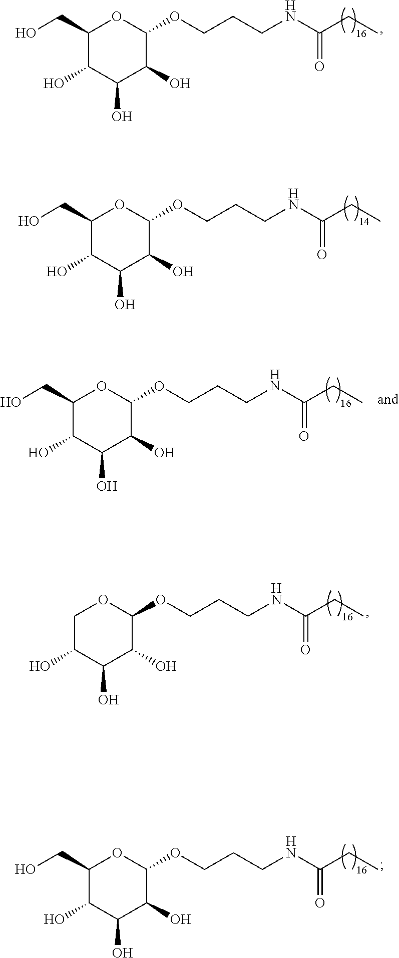
and
at least one α-cyclodextrin.

7. The particles according to claim 1, wherein the particles have a hydrodynamic diameter ranging from 10 nm to 10 μm and/or a number average diameter D_{TEM} ranging from 10 nm to 10 μm.

8. The particles according to claim 1 further comprising at least one molecule of therapeutic or cosmetic interest.

9. A method for preparing particles according to claim 1 comprising a step of mixing:
at least one compound of formula Chem. (I) as defined in claim 1;
at least one cyclodextrin; and
optionally a molecule of therapeutic or cosmetic interest.

10. The method according to claim 9, wherein the compound of formula Chem. (I) and the cyclodextrin are introduced in a molar ratio compound of formula Chem. (I):cyclodextrin ranging from 1:1 to 1:20.

11. The method according to claim 9, wherein the step of mixing is carried out in water and/or a water-miscible solvent.

12. A cosmetic or pharmaceutical composition comprising particles according to claim 1 at least one cosmetically or pharmaceutically acceptable excipient.

13-14. (canceled)

15. The particles according to claim 1, wherein the at least one cyclodextrin is a α-cyclodextrin.

16. The particles according to claim 8, wherein the molecule of therapeutic or cosmetic interest is selected from the group consisting of vitamins, antimicrobial peptides, antiviral agents, and antibiotics.

17. The method according to claim 11, wherein the step of mixing is carried out in water.

18. The cosmetic or pharmaceutical composition according to claim 12, wherein the cosmetic or pharmaceutical composition is suitable for topical application.

19. A method for a sustained release of a molecule of therapeutic or cosmetic interest in a subject in need thereof comprising a step of administering the particles as defined in claim 8 to the subject in need thereof.

20. The method according to claim 19, wherein the subject is suffering from a bacterial infection.

21. A method for treating a disease comprising a step of administering the particles as defined in claim 1 to a patient in need thereof.

22. The method according to claim 21, wherein the disease is a bacterial infection.
